(19) United States
(12) Patent Application Publication
(10) Pub. No.: US 2024/0156415 A1
(43) Pub. Date: May 16, 2024

(54) DIAGNOSIS SUPPORT SYSTEM AND DIAGNOSIS SUPPORT APPARATUS

(71) Applicant: Canon Medical Systems Corporation, Otawara-shi (JP)

(72) Inventors: Tomoko TAKAYAMA, Tokyo (JP); Atsuko SUGIYAMA, Nasushiobara (JP); Yusuke KANO, Nasushiobara (JP); Katsuhiko FUJIMOTO, Saitama (JP); Hayato OKUMIYA, Nasushiobara (JP); Mariko SHIBATA, Nasushiobara (JP)

(73) Assignee: Canon Medical Systems Corporation, Otawara-shi (JP)

(21) Appl. No.: 18/460,945

(22) Filed: Sep. 5, 2023

(30) Foreign Application Priority Data
Sep. 5, 2022 (JP) 2022-140550
Sep. 30, 2022 (JP) 2022-158187
Sep. 4, 2023 (JP) 2023-143186

(51) Int. Cl. A61B 5/00; G16H 10/60

(52) U.S. Cl. CPC A61B 5/7271; A61B 5/742; G16H 10/60

(57) ABSTRACT

A diagnosis support system includes an acquirer, a calculator, and a display controller. The acquirer acquires diagnosis data. The calculator calculates, based on the medical data, a feature amount in the medical data for a predetermined period. The display controller causes the displayer to display the medical data in a format according to the feature amount.

### CROSS-REFERENCE TO RELATED APPLICATIONS

This application is based upon and claims the benefit of priority from Japanese Patent Application No. 2022-140550, filed Sep. 5, 2022, Japanese Patent Application No. 2022-158187, filed Sep. 30, 2022, and Japanese Patent Application No. 2023-143186, filed Sep. 4, 2023, the entire contents of all of which are incorporated herein by reference.

### FIELD

Embodiments described herein relate generally to a diagnosis support system and a diagnosis support apparatus.

### BACKGROUND

Recent team medical-care (Team Approach) has increased occasions in which a plurality of healthcare professionals (Multidisciplinary Team) perform a medical treatment on one patient, and the importance of such occasions are increasing. Team medical-care requires communications between a patient and people of various professionals, thereby being able to collect various pieces of information on the patient.

However, simply gathering collected pieces of information leads to information overload which involves a problem wherein confirmation of information takes much time and hard to access appropriate data. Furthermore, this may make it difficult to determine which piece of information is important. It is known that patient's views, preferences, values and wishes depend on who are talking to, a place and a situation in which the patient talks, information that the patient has, a situation of patient and so on. For this reason, in considering, selecting, or determining a treatment policy for a patient, it is important to share pieces of information on the patient collected from various viewpoints and multifaced grasp the patient's views, preferences, values and wishes. The above circumstances demand a system that collects and integrates pieces of information obtained by various medical staff and professionals through their interactions with a patient care pathway from various dimensions, and supports a diagnosis, medical treatment or care using the collected pieces of information on various aspects of the patient.

### BRIEF DESCRIPTION OF THE DRAWINGS

FIG. 1 is a diagram showing an example of a configuration of a diagnosis support system according to a first embodiment.

FIG. 2 is a diagram showing an example of a system connected to a medical database according to the first embodiment.

FIG. 3 is a flowchart showing an example of a processing procedure of diagnosis support processing performed by the diagnosis support system according to the first embodiment.

FIG. 4 is a diagram showing an example of a diagnosis support screen displayed on a displayer according to the first embodiment.

FIG. 5 is a diagram showing an example of a detailed item displayer displayed on the diagnosis support screen according to the first embodiment.

FIG. 6 is a diagram showing an example of a method of displaying display items shown in FIG. 4 and FIG. 5.

FIG. 7 is a diagram showing a related information displayer displayed on the diagnosis support screen according to the first embodiment.

FIG. 8 is a diagram showing a modification of the related information displayer displayed on the diagnosis support screen according to the first embodiment.

FIG. 9 is a diagram showing a modification of the related information displayer displayed on the diagnosis support screen according to the first embodiment.

FIG. 10 is a diagram showing a modification of the related information displayer displayed on the diagnosis support screen according to the first embodiment.

FIG. 11 is a diagram showing a modification of the related information displayer displayed on the diagnosis support screen according to the first embodiment.

FIG. 12 is a diagram showing a modification of the related information displayer displayed on the diagnosis support screen according to the first embodiment.

FIG. 13 is a diagram showing a modification of the detailed item displayer displayed on the diagnosis support screen according to the first embodiment.

FIG. 14 is a diagram showing a modification of a major item displayer displayed on the diagnosis support screen according to the first embodiment.

FIG. 15 is a diagram showing a modification of the major item displayer displayed on the diagnosis support screen according to the first embodiment.

FIG. 16 is a diagram showing an example of a detailed item displayer displayed on the diagnosis support screen according to the first embodiment.

FIG. 17 is a diagram showing a modification of the related information displayer displayed on the diagnosis support screen according to the first embodiment.

FIG. 18 is a diagram showing a modification of the related information displayer displayed on the diagnosis support screen according to the first embodiment.

FIG. 19 is a diagram showing a modification of the related information displayer displayed on the diagnosis support screen according to the first embodiment.

FIG. 20 is a diagram showing a modification of the related information displayer displayed on the diagnosis support screen according to the first embodiment.

FIG. 21 is a diagram showing a modification of the related information displayer displayed on the diagnosis support screen according to the first embodiment.

FIG. 22 is a diagram showing a modification of the related information displayer displayed on the diagnosis support screen according to the first embodiment.

FIG. 23 is a diagram showing a modification of the related information displayer displayed on the diagnosis support screen according to the first embodiment.

FIG. 24 is a diagram showing a modification of the related information displayer displayed on the diagnosis support screen according to the first embodiment.

FIG. 25 is a diagram showing a modification of the related information displayer displayed on the diagnosis support screen according to the first embodiment.

FIG. 26 is a diagram showing a modification of the related information displayer displayed on the diagnosis support screen according to the first embodiment.

FIG. 27 is a diagram showing a modification of the related information displayer displayed on the diagnosis support screen according to the first embodiment.

FIG. 28 is a diagram showing a modification of the related information displayer displayed on the diagnosis support screen according to the first embodiment.

FIG. 29 is a diagram showing a modification of the related information displayer displayed on the diagnosis support screen according to the first embodiment.

FIG. 30 is a diagram showing a modification of the related information displayer displayed on the diagnosis support screen according to the first embodiment.

FIG. 31 is a diagram showing a modification of the related information displayer displayed on the diagnosis support screen according to the first embodiment.

FIG. 32 is a diagram showing a modification of the related information displayer displayed on the diagnosis support screen according to the first embodiment.

FIG. 33 is a diagram showing a modification of the related information displayer displayed on the diagnosis support screen according to the first embodiment.

FIG. 34 is a block diagram showing an example of a configuration of a diagnosis support system according to a second embodiment.

FIG. 35 is a diagram for explaining one example of processing of registering diagnosis information in a medical database according to the second embodiment.

FIG. 36 is a diagram showing an example of a category specifying table according to the second embodiment.

FIG. 37 is a diagram showing an example of displaying of diagnosis information according to the second embodiment.

FIG. 38 is a diagram showing an example of displaying of diagnosis information according to the second embodiment.

FIG. 39 is a diagram showing an example of displaying of diagnosis information according to the second embodiment.

FIG. 40 is a diagram showing an example of information obtained by visualizing a first fluctuation value according to the second embodiment.

FIG. 41 is a diagram showing an example of displaying of diagnosis information according to the second embodiment.

FIG. 42 is a diagram showing an example of displaying of diagnosis information according to the second embodiment.

FIG. 43 is a diagram showing an example of information obtained by visualizing a second fluctuation value according to the second embodiment.

FIG. 44 is a diagram showing an example of displaying of diagnosis information according to the second embodiment.

FIG. 45 is a diagram showing an example of displaying of diagnosis information according to the second embodiment.

FIG. 46 is a diagram showing an example of displaying of diagnosis information according to the second embodiment.

FIG. 47 is a diagram showing an example of displaying of diagnosis information according to the second embodiment.

FIG. 48 is a flowchart showing an example of processing performed by a diagnosis support apparatus according to the second embodiment.

FIG. 49 is a diagram showing an example of displaying of diagnosis information according to a modification of the second embodiment.

FIG. 50 is a diagram showing an example of displaying of diagnosis information according to the second embodiment.

### DETAILED DESCRIPTION

In general, according to one embodiment, a diagnosis support system includes an acquirer, a calculator, and a display controller. The acquirer acquires diagnosis data. The calculator calculates, based on the medical data, a feature amount in the medical data for a predetermined period. The display controller causes the displayer to display the medical data in a format according to the feature amount.

Hereinafter, embodiments of a diagnosis support system and a diagnosis support apparatus will be described in detail with reference to the accompanying drawings. In the following description, structural elements having approximately the same function and configuration will be assigned the same reference symbol, and a repeat description will be given only where necessary.

### First Embodiment

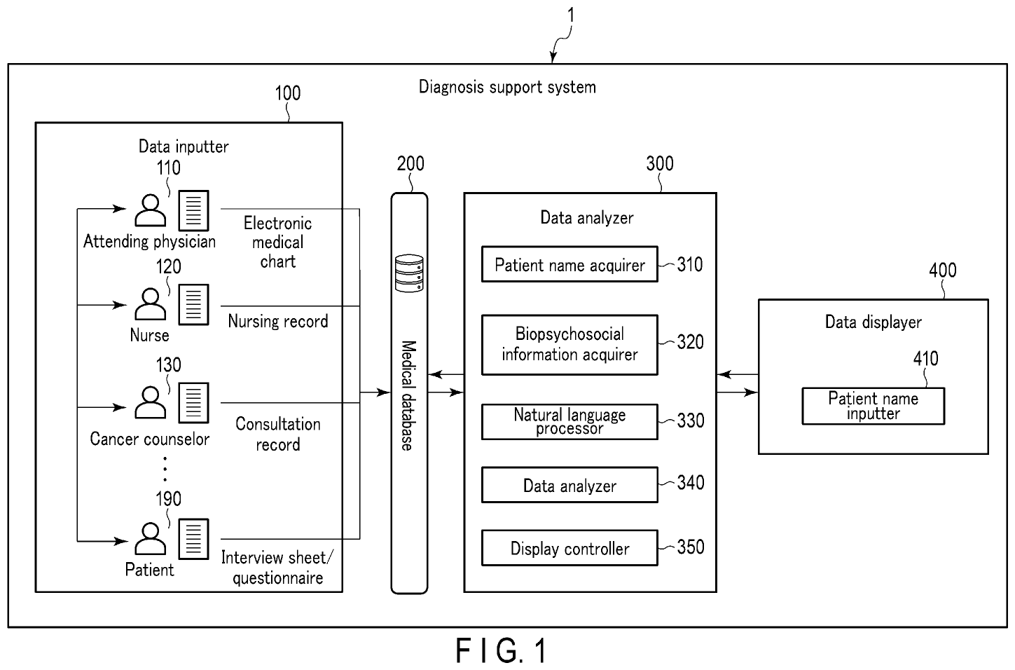

FIG. 1 is a diagram showing a configuration of an example of the diagnosis support system 1 according to the first embodiment. The diagnosis support system 1 according to the present embodiment includes a data inputter 100, a medical database 200, a data analyzer 300, and a data displayer 400. The data inputter 100, the medical database 200, the data analyzer 300, and the data displayer 400 are connected to each other via a network. The network is, for example, a Local Area Network (LAN). The connection to the network may be established in either a wired or wireless manner. Furthermore, as long as security is ensured by means of a Virtual Private Network (VPN), etc., a line to be connected is not necessarily a LAN. A connection to the Internet or other public communication network may be established.

In the data inputter 100, a plurality of various medical records is input by a plurality of healthcare professionals. Examples of the medical records include an electronic chart, a nursing record chart, a consultation record, an interview sheet, a questionnaire, etc. In each medical record, information on a patient's biomedical dimension, information on his or her psychological dimension, and information on his or her social dimension are recorded in a mixed manner. The medical record may be text data in which sentences are recorded, voice data in which a patient's voice, etc. is recorded, or video data in which an interview, etc., is recorded.

The data inputter 100 is constituted by a plurality of terminals operated by a healthcare professional. The data inputter 100 includes, for example, an electronic medical chart system 110 for inputting electronic medical charts, a nursing work support system 120 for inputting nursing record charts, a consultation record system 130 for inputting consultation records, and a patient daily life information system 190 for inputting an interview sheet and a questionnaire. In the patient daily life information system 190, for example, a patient and his or her family inputs information into a digitized interview sheet and questionnaire, and the input information is transmitted to the medical database 200. The patient daily life information system 190 may be a terminal configured to scan and digitize an interview sheet and a questionnaire written on paper. The data inputter 100 transmits the input medical record to the medical database 200.

The medical database 200 is a database configured to store a medical record input with the data inputter 100. The medical database 200 transmits the medical record to the data analyzer 300 in response to a command therefrom. The medical database 200 may also be referred to as a medical database (medical information DB).

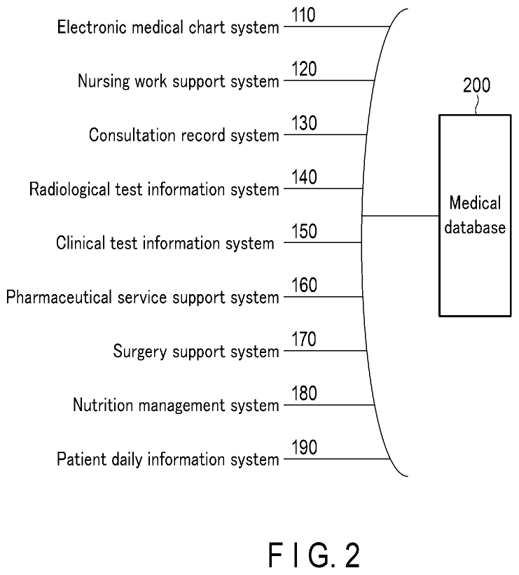

The medical database 200 may be connected to a system other than the data inputter 100. FIG. 2 is a diagram showing an example of systems connected to the medical database 200. As shown in FIG. 2, the medical database 200 may be connected to not only the data inputter 100 described above but also a radiological test information system 140, a clinical test information system 150, a pharmaceutical service support system 160, a surgery support system 170, a nutrition management system 180, and a patient daily life information system 190. The patient daily life information system 190 is, for example, an electronic patient reported outcome system (ePRO system). The medical database 200 acquires and stores various types of medical records from the radiological test information system 140, the clinical test information system 150, the pharmaceutical service support system 160, the surgery support system 170, the nutrition management system 180, and the patient daily life information system 190. Not all of the above systems may be connected to the medical database 200.

The data analyzer 300 acquires medical data including various medical records, performs analysis processing on the medical data, extracts medical records related to psychosocial items from the medical data, and calculates the information amount for the psychosocial items. The data analyzer 300 then causes the data displayer 400 to display thereon the psychosocial items in a mode according to the information amount. An apparatus for realizing the functions of the data analyzer 300 is an example of the diagnosis support apparatus. The analysis processing is, for example, natural language processing. The present embodiment will describe the case of using natural language processing as the analysis processing; however, another processing may be used. Natural language processing may be manually performed, and a result of the natural language processing may also be manually corrected.

The data displayer 400 displays a display screen (hereinafter referred to as a diagnosis support screen) for supporting a diagnosis of a patient to be diagnosed. The data displayer 400 is realized by, for example, a device having a liquid crystal display or a cathode ray tube (CRT) display. The data displayer 400 is formed as a terminal for allowing medical records to be input into the electronic medical chart system 110, the nursing work support system 120, and the consultation record system 130. The data displayer 400 may be another terminal such as a computer, a tablet terminal, or a smartphone terminal.

The data displayer 400 may be a terminal for use in an online conference system, etc. In this case, the data displayer 400 displays a result of the analysis on a shared dashboard that allows therein a plurality of participants to write sentences under a teleconference system, for example. Furthermore, the data displayer 400 may be a terminal connected to a multidisciplinary meeting application.

Furthermore, the data displayer 400 displays a patient name inputter 410. A name of a patient to be diagnosed (hereinafter referred to as a patient name) is input into the patient name inputter 410. The data displayer 400 transmits a patient name input into the patient name inputter 410 to the data analyzer 300. The patient name is an example of the patient information. Furthermore, the data displayer 400 displays the result of analysis transmitted from the data analyzer 300.

Next, a configuration and a function of the data analyzer 300 will be described in detail.

The data analyzer 300 includes a memory and a processing circuit, both not shown. For example, the data analyzer 300 will be described as a single device that performs a plurality of functions; however, these functions may be performed by separate devices. For example, the functions performed by the data analyzer 300 may be mounted on different console devices or work station devices in a distributed manner.

The memory is a storage device such as a hard disk drive (HDD), a solid-state drive (SSD), or an integrated circuit which stores various items of information. Other than the HDD and SSD, the memory may be a portable storage medium such as a compact disc (CD), a digital versatile disc (DVD), a flash memory, etc. Alternatively, the memory may be a driver that writes and reads various pieces of information to and from a semiconductor memory such as a flash memory, a random-access memory (RAM), etc. The storage area of the memory may be in the data analyzer 300 or in an external storage device connected via a network.

The memory stores a program to be performed by the processing circuit, various types of data to be used for the processing by the processing circuit, etc. Such a program may be installed in advance in a computer from a network or a non-transitory computer-readable storage medium, for example, so that the program will cause the computer to implement the respective functions of the processing circuit. Various types of data handled in this disclosure are typically digital data. The memory is an example of the storage.

The processing circuit controls the overall operation of the data analyzer 300. The processing circuit is a processor configured to, upon calling up and performing a program from the memory, realize functions of a patient name acquirer 310, a biopsychosocial information acquirer 320, a natural language processor 330, a data analyzer 340, and a display controller 350. The patient name acquirer 310, the biopsychosocial information acquirer 320, the natural language processor 330, the data analyzer 340, and the display controller 350 may be respectively referred to as a patient name acquisition function, a biopsychosocial information acquisition function, a natural language processing function, a data analyzing function, and a display controlling function.

Meanwhile, the functions of the patient name acquirer 310, the biopsychosocial information acquirer 320, the natural language processor 330, and the data analyzer 340 may be realized by the single processing circuit, or may be configured by a combination of a plurality of independent processors and the functions may be realized by the processors performing the programs. The functions of the patient name acquirer 310, the biopsychosocial information acquirer 320, the natural language processor 330, the data analyzer 340, and the display controller 350 may be realized as individual hardware circuits, respectively. Furthermore, the functions of the patient name acquirer 310, the biopsychosocial information acquirer 320, the natural language processor 330, the data analyzer 340, and the display controller 350 may be mounted on a cloud server. The above description of the functions performed by the processing circuit is applicable to the embodiments and modifications described below.

The data analyzer 300 performs multiple functions with a single console; however, these functions may be implemented by separate devices. For example, the functions of the processing circuit may be mounted on different devices or servers on a network in a distributed manner.

The term “processor” used herein refers to, for example, a central processing unit (CPU) or a graphics processing unit (GPU), or a circuit such as an application-specific integrated circuit (ASIC), a programmable logic device (such as a simple programmable logic device (SPLD)), a complex programmable logic device (CPLD), a field programmable gate array (FPGA)), etc. The function corresponding to a program is directly incorporated into a circuit of the processor as a logic circuit, instead of being stored in the storage circuit. In the case of the processor being a CPU, for example, the processor realizes a function by reading and performing a program stored in a memory. On the other hand, in the case of the processor being, for example, an ASIC, instead of a program being stored in the memory, a corresponding function may be directly incorporated as a logic circuit into the circuit of the processor. In this case, the processor reads the programs incorporated into its circuit and performs them to realize the functions. The embodiments herein do not limit each processor to a single circuit-type processor. Multiple independent circuits may be combined and integrated as one processor to realize the intended functions. Furthermore, multiple components or features as given in FIG. 1 may be integrated as one processor to realize the respective functions. The above description of the “processor” is applicable to the subsequent embodiments and modifications.

The patient name acquirer 310 acquires a patient name input via the patient name inputter 410 of the data displayer 400 from the data displayer 400.

Based on the patient name acquired by the patient name acquirer 310, the biopsychosocial information acquirer 320 acquires medical data on a patient to be diagnosed from the medical database 200. Hereinafter, a patient to be diagnosed will be simply referred to as a patient. The medical data includes medical records of a patient acquired at a plurality of points in time and the medical records are of a plurality of types. In this respect, the biopsychosocial information acquirer 320 reads, as a medical record, for example, an electronic medical chart, a nursing record, a consultation record, an interview sheet, a questionnaire, etc. about the patient from the medical database 200. The processing circuit that realizes the functions of the biopsychosocial information acquirer 320 is one example of the acquirer that acquires medical data including medical records acquired at a plurality of points in time. The medical record may also be referred to as medical information.

The natural language processor 330 performs natural language processing on each medical record acquired by the biopsychosocial information acquirer 320. The natural language processor 330 extracts, for each preset display item, a description related to a corresponding display item from the medical data. Such a description is extracted in unit of a medical record, in unit of a sentence included in the medical record, or in unit of a paragraph included in the medical record. An example of unit of a paragraph extracted from a document will be described. Sentences regarding a patient are, if they are in a form called a “progress note” or “SOAP charting”, described in such a manner as to be organized by categories such as subjective information (S), objective information (O), assessment (A), planning (P), etc. In such a case, paragraphs may be extracted while each category is regarded as one paragraph. The extracted description is associated with a corresponding display item, as its related information. The display items can be classified into a biomedical item and a psychosocial item. Accordingly, the natural language processor 330 extracts a description related to the psychosocial item from the medical data based on a result of the natural language processing, and associates, as related information, the extracted description with the psychosocial item. The processing circuit that realizes the functions of the natural language processor 330 is one example of a calculator. Herein, as an example, a case of extracting a description in unit of a sentence will be described in detail. In such a case, the natural language processor 330 first divides text data included in a medical record into sentences. In a case of the medical record being audio data or video data, the natural language processor 330 converts medical data into text data and then divides the text data included in the medical record for each text data. The natural language processor 330 then determines a related display item for each sentence and associates information indicative of the related display item with each corresponding sentence. Thereafter, the natural language processor 330 extracts, for each display item, a sentence associated with information indicative of a corresponding display item, as a sentence related to the corresponding display item.

The function of the natural language processor 330 may be mounted on each of the systems 110 to 190 of the data inputter 100. For example, in the case where a medical record is stored in each of the systems 110 to 190, text data included in the medical record may be divided into text units, and a function of determining a related display item for each text may be executed by each of the systems 110 to 190. Furthermore, a related display item for each text included in a medical record may be input by a user. Yet further, when the medical record is stored or before a description related to a display item is extracted, a determination result of a related display item for each text may be presented to a user and the display item may be verified or corrected by the user.

The natural language processing can use various known techniques. For example, the natural language processing can use known analysis techniques such as Word2Vec, Doc2Vec, Sent2Vec, bidirectional encoder representations from transformers (BERT), language processing using the context vector similarity, etc.

The display item is an item freely selected and displayed on the diagnosis support screen. The display item is an item for allowing holistic understanding of a patient through medical treatment and care. The display items can be classified into, for example, a group of medical indications, a group of patient's views and preferences, a group of QOL, a group of contextual features, etc. Examples of the display items include items indicated by a questionnaire about ease of living and a scientific, reliable, and reasonable scale for measuring a health-related quality of life (HRQOL). For example, as the display items, pieces used in a questionnaire about ease of living, a QOL scale for breast cancer patients, FACT-B, etc. are usable. Other than the above, examples of the display item my include presented pieces used in, for example, European Organization for Research and Treatment of Cancer (EORTC) QLQ-BR23, a questionnaire for measuring the quality of life of breast cancer patients, an Integrated Palliative Care Outcome Scale (IPOS), which is an evaluation of the quality of palliative care by patients and healthcare professionals, a Support Team Assessment Schedule-Japanese version (STAS-J), which is an evaluation scale in hospice and palliative care, an Edmonton Symptom Assessment Scale (ESAS), a Distress Thermometer (DT), a Distress and Impact Thermometer, a 5th vital sign, etc. In addition, items unique to the facility in use may be used as the display items. Examples of the display items may include a combination of more than one of the plurality of tools described above. The display items may be preset or may be set by a user at the time of use. Examples of the display items may include a word that appears frequently in a medical record in text format and a word repeated or emphasized loudly by a patient in a description in audio or video form.

As described above, the display items can be classified into a biomedical item and a psychosocial item.

The biomedical item is an item related to the medical dimension or biological dimension of a patient. Examples of the biomedical item include “medical indication”, “physical status”, etc. “Medical indication” can be classified into more specific items such as “tumor size”, “lymph node metastasis”, “presence or absence of metastasis”, “subtype”, “pathological grade”, “co-morbidities”, etc. In addition, the “physical status” include “pain”, “menopause-like symptoms”, “skin troubles”, “dental trouble”, “urination/defecation trouble”, “appetite”, “sleep”, “nausea/vomiting”, “feeling of fatigue/tiredness”, “lymphoedema”, etc.

The psychosocial item is an item related to the psychological dimension and social dimension of a patient. Examples of the psychosocial item include “patient's views and preferences”, “contextual features”, “concern/worry”, “mental status”, etc. “Patient's views and preferences” can be classified into more specific items such as “knowing life expectancy”, “focus on appearance”, “treatment”, “focus on hospital visit period”, “palliative care”, “clinical study”, etc. “Contextual features” can be classified into more specific items such as “hobby”, “family intentions”, “child care/nursing care”, “ease of hospital visit”, “insurance”, “financial situation”, etc. “Concern/worry” can be classified into more specific items such as “symptoms, care, and treatment”, “economy and social services”, “daily life”, “family”, “job”, “hospital visit and admission”, “support, palliative care, terminal stage”, “appearance”, etc. In addition, “mental status” can be classified into more specific items such as “depressed feeling”, “difficulty of life”, etc.

The data analyzer 340 calculates, based on the medical data, a feature amount in the medical data for a predetermined period. In the present embodiment, the amount of related information (hereinafter referred to as an “information amount”) is used as a feature amount. The data analyzer 340 according to the present embodiment calculates the information amount for each of the biomedical item and the psychosocial item by calculating the information amount linked by the natural language processor 330 for each display item. The data analyzer 340 outputs the calculated information amount as a result of the analysis to the display controller 350. The processing circuit that realizes the functions of the data analyzer 340 is one example of the calculator.

The information amount is the number of descriptions extracted as related information. As the information amount, for example, the number of texts extracted as the related information, the number of paragraphs extracted as the related information, the number of medical records extracted as the related information, and the number of data types of the medical records extracted as the related information may be used. The number of paragraphs including one or more texts extracted as the related information may be used as the information amount. Examples of the data type include types in a storage format such as text data, voice data, video data, etc. In such a case, the number of data types (text data, voice data, and video data) included in medical records associated as related information for each display item is calculated as the information amount. Meanwhile, examples of the data type may include types of documents such as an electronic medical chart, a nursing record, a consultation record, an interview sheet, etc.

The information amount may be calculated using the number of staff members who have entered descriptions extracted as the related information. In this case, for example, the number of nurses who have entered nursing records associated as related information is used as the information amount.

Alternatively, the information amount may be calculated using a similarity between a plurality of descriptions extracted as related information. This case has a setting in which, for example, the information amount becomes greater in value as the similarity decreases. Alternatively, the information amount may be calculated using, with respect to a reference medical record, a total value or an average value of similarities of other multiple medical records.

Alternatively, the information amount may be calculated using the number of letters included in a description extracted for a display item.

In the above calculation method, in the case of a plurality of descriptions associated with a display item, a total value, an average value, etc., of parameters for use may be used as appropriate. Furthermore, the information amount may be calculated by combining the plurality of parameters described above.

The display controller 350 causes the data displayer 400 to display various pieces of information. For example, the display controller 350 generates screen data for causing the data displayer 400 to display the diagnosis support screen, and outputs the generated screen data to the data displayer 400. Furthermore, the display controller 350 causes the displayer to display the medical data in a format according to the feature amount. The display controller 350 according to the present embodiment visualizes the presence or absence of related information for a display item and the information amount of the related information and causes the data displayer 400 to display them, thereby causing the data displayer 400 to display the psychosocial item in a mode according to the information volume. The display controller 350 causes the data displayer 400 to display the medical record extracted as the related information along with time-series information.

The data displayer 400 displays the display screen data acquired from the display controller 350. The diagnosis support screen displays, for example, the biomedical items and the psychosocial items in a collective manner for each group of related items. The data displayer 400 visualizes and displays thereon the presence or absence of related information for a display item and the information amount of the related information. For example, the information amount can be displayed in such a manner that allows visual discrimination by changing the size, color, darkness, shape, font, etc., of an icon or characters indicative of a corresponding display item according to the information amount. Furthermore, the display items may be classified into levels such as “large” and “small” by performing threshold determination on a value of the information amount, and may be displayed in a mode according to the classified stage. The classification levels may be two or more. Each display item may be displayed using a pixel value, a color value, and a size according to the value of the information amount. As long as the information amount between the display items is visually recognizable, the display items may be displayed by changing whether or not the icon blinks, the blinking speed, the amount of light emitted, etc., in addition to the color or size. Depending on the information amount, a warning sound may be generated to call the attention of healthcare professionals, etc.

Furthermore, a change over time in a parameter related to a description associated with a corresponding display item may be displayed on the diagnosis support screen. In such a case, a multi-views of a patient can be presented, thereby making it easier for a user to find items to check. Furthermore, a display mode of a display item may be changed according to the width of fluctuation between descriptions associated with the display item. The width of fluctuation is a value indicative of the degree of variation in matters related to a display item between the plurality of descriptions associated with a corresponding display item. For example, in the plurality of medical records associated with the display item, the width of fluctuation increases in the case where a patient gives different answers to the display item. In this case, the data analyzer 340 calculates the width of fluctuation of the psychosocial items among the plurality of medical records extracted as related information, and the display controller 350 displays the psychosocial items according to the width of fluctuation.

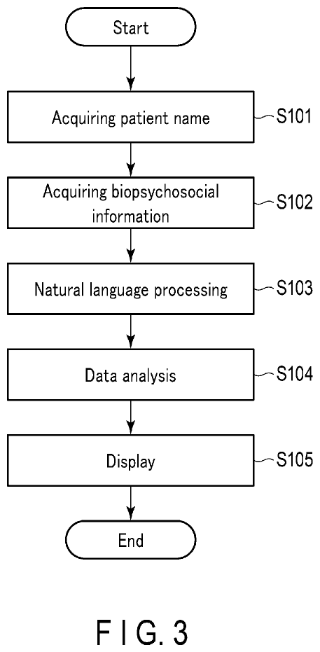

Next, the operation of the diagnosis support processing performed by the diagnosis support system 1 will be described. The diagnosis support processing is processing for visualizing and displaying the information amount for each display item on the diagnosis support screen displayed on data the data displayer 400. FIG. 3 is a flowchart showing an example of a processing procedure of the diagnosis support processing. The following will describe an example case in which items used in a questionnaire about ease of living are adopted as the display items. A processing procedure of each processing described below is merely an example, and each processing may be suitably changed where possible. Omission, replacement, and addition of a step in the processing procedures described hereinafter can be made as appropriate, in accordance with an actual situation where the present embodiment is realized.

### (Diagnosis Support Processing)

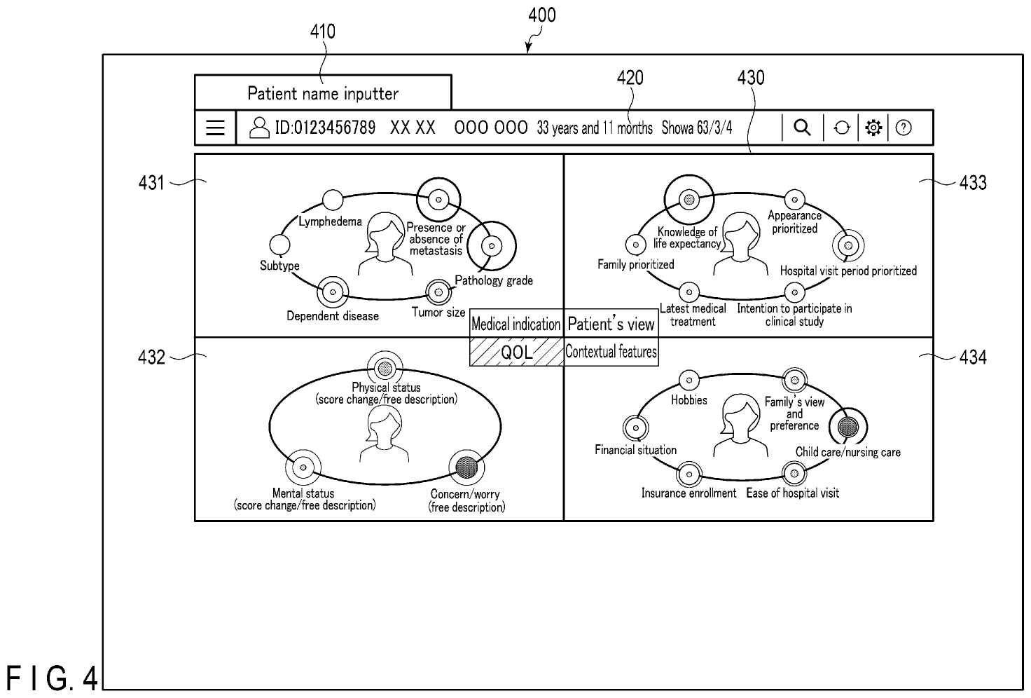

The diagnosis support processing is initiated while the diagnosis support screen is displayed on the data displayer 400. FIG. 4 is a diagram showing an example of the diagnosis support screen displayed on the data displayer 400. As shown in FIG. 4, the diagnosis support screen includes the patient name inputter 410. Information for identifying a patient to be diagnosed is input into the patient name inputter 410. For example, a patient name, a patient ID, etc., are input by a user into the patient name inputter 410. The patient name may be input via a terminal operated by a healthcare professional, such as the electronic medical chart system 110, the nursing work support system 120, the consultation record system 130, etc.

### (Step S101)

In response to the patient name being input into the patient name inputter 410, the patient name acquirer 310 acquires patient name input data in the patient name inputter 410 from the data displayer 400. The display controller 350 causes the diagnosis support screen to display the patient information displayer 420. The patient information displayer 420 shows a patient's basic information input into the patient name inputter 410. As the basic information, for example, a patient name, a patient ID, an age, data of birth, gender, etc., are displayed.

### (Step S102)

Next, based on the patient name acquired in step S101, the biopsychosocial information acquirer 320 extracts and acquires medical records of a patient from the medical database 200. In the medical records, for example, an electronic medical chart, a nursing record, a consultation record, an interview sheet, a questionnaire, etc., about the patient are collected from the medical database 200.

### (Step S103)

Next, the natural language processor 330 performs the natural language processing on each medical record acquired in step S102, and associates each display item with a description (hereinafter referred to as a “related description”) related to the display item concerned. In this processing, the natural language processor 330 calculates a similarity to each display item for each text included in each medical record, and associates each of the display items having a similarity equal to or greater than a predetermined value with a related description of each display item. At this time, medical records in an audio data format or video data format are converted into text using known audio analysis software or video analysis software, and the natural language processing is performed on sentences obtained by text conversion.

This associates each display item with its related description. The number of related descriptions associated with one text may be one or plural. Furthermore, the number of related descriptions associated with one display item may be one or plural.

### (Step S104)

Next, the data analyzer 340 calculates, for each display item, the information amount of the related descriptions associated with the display item concerned. The data analyzer 340 calculates, as the information amount, for example, the number of texts associated as related descriptions. However, this is not a limitation, and the data analyzer 340 may calculate the information amount based on the similarity between the plurality of extracted related descriptions.

### (Step S105)

Next, the display controller 350 causes the diagnosis support screen to display the information displayer 430. The information displayer 430 during a normal time displays thereon a major item display screen. On the major item display screen, the plurality of display items is grouped into a plurality of groups (major items) and displayed. In the example shown in FIG. 4, the plurality of display items is classified into four major items: “medical indication”, “QOL”, “patient's views and preferences”, and “contextual features”. The information displayer 430 displays thereon a first displayer 431 on which medical items classified as the major item of “medical indication” are displayed, a second displayer 432 on which medical items classified as the major item of “QOL” are displayed, a third displayer 433 on which medical items classified as the major item of “patient's view and preference” are displayed, and a fourth displayer 434 on which medical items classified as the major item of “contextual features” are displayed. The major items may be referred to as a “category”.

The first displayer 431 displays thereon the display items related to “biomedical information (medical indication)”. The display items related to “medical indication” include, for example, “lymphedema”, “presence or absence of metastasis”, “tumor size”, and “pathological grade”. The second displayer 432 displays thereon the display items related to “QOL”. The display items related to “QOL” include, for example, “physical status”, “mental status”, “concern/worry”. The third displayer 433 displays thereon the display items related to “patient's view and preference”. The display items related to “patient's view and preference” include, for example, “family importance”, “knowing life expectancy”, “emphasis on appearance”, “clinical study participation intentions”, etc. The fourth displayer 434 displays thereon the display items related to the “contextual features”. The display items related to “contextual features” include, for example, “subscription to insurance”, “financial situation”, “family's view and preference”, and “child care/nursing care”.

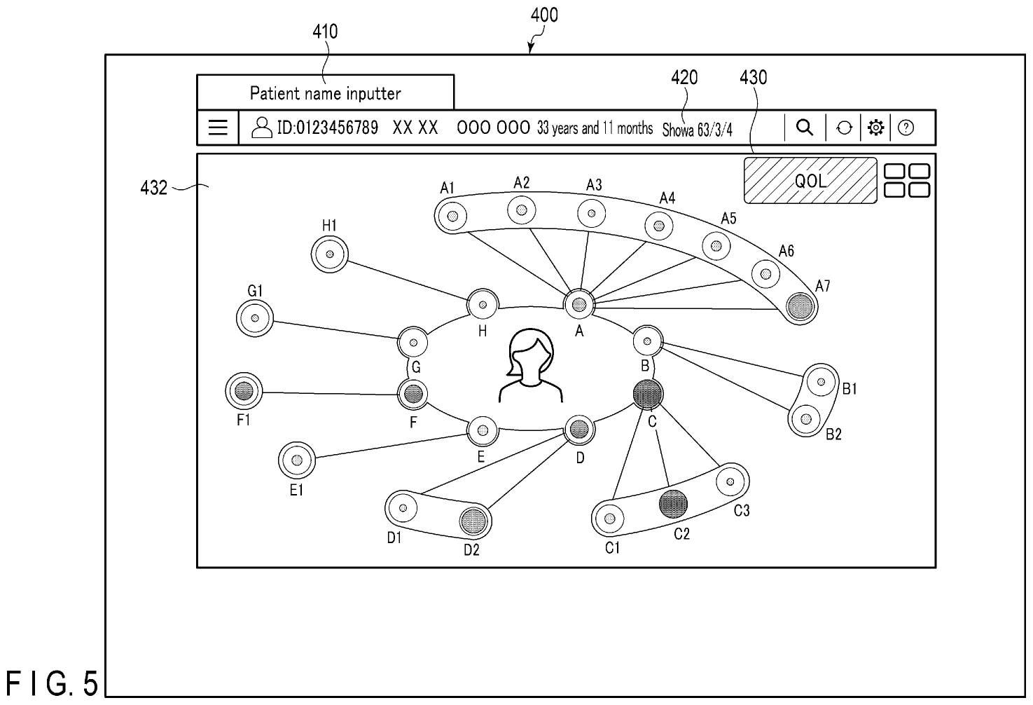

In response to one of the major items displayed on the first to fourth displayers 431 to 434 being selected, a detailed item display screen in which the selected displayer is enlarged is displayed on the information displayer 430. FIG. 5 shows an example of the detailed item display screen. FIG. 5 shows an example in the case in which the second displayer 432 corresponding to the major item of “QOL” is selected. The information displayer 430 displays thereon display items obtained by further classifying each of the display items displayed on the second displayer 432 shown in FIG. 4.

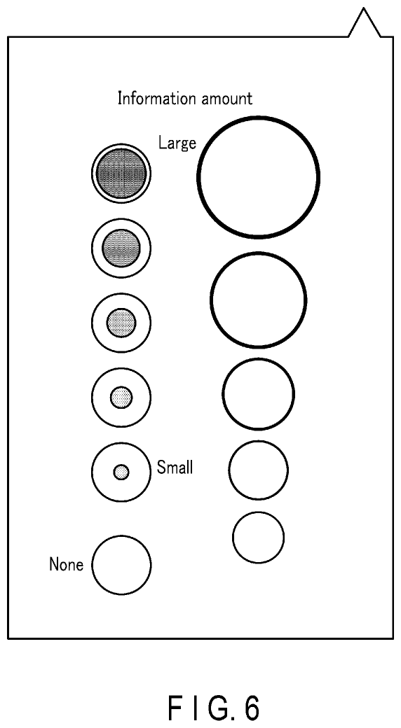

Furthermore, the display controller 350 visualizes the information amount calculated in step S104 and displays it on the data displayer 400. FIG. 6 is a diagram for explaining a method of visualizing the information amount applied to FIG. 4 and FIG. 5. As shown in FIG. 6, in FIG. 4 and FIG. 5, the information amount of the display items is visualized and displayed in such a manner that a black circle included in each of the icons of the display items becomes larger as the information amount increases. Furthermore, in FIG. 4, the information amount is displayed in such a manner that an outer frame of each of the icons of the display items becomes larger and darker as the information amount increases. By checking the icons of the display items, a user can visually grasp an item with the large information amount and easily grasp an important item.

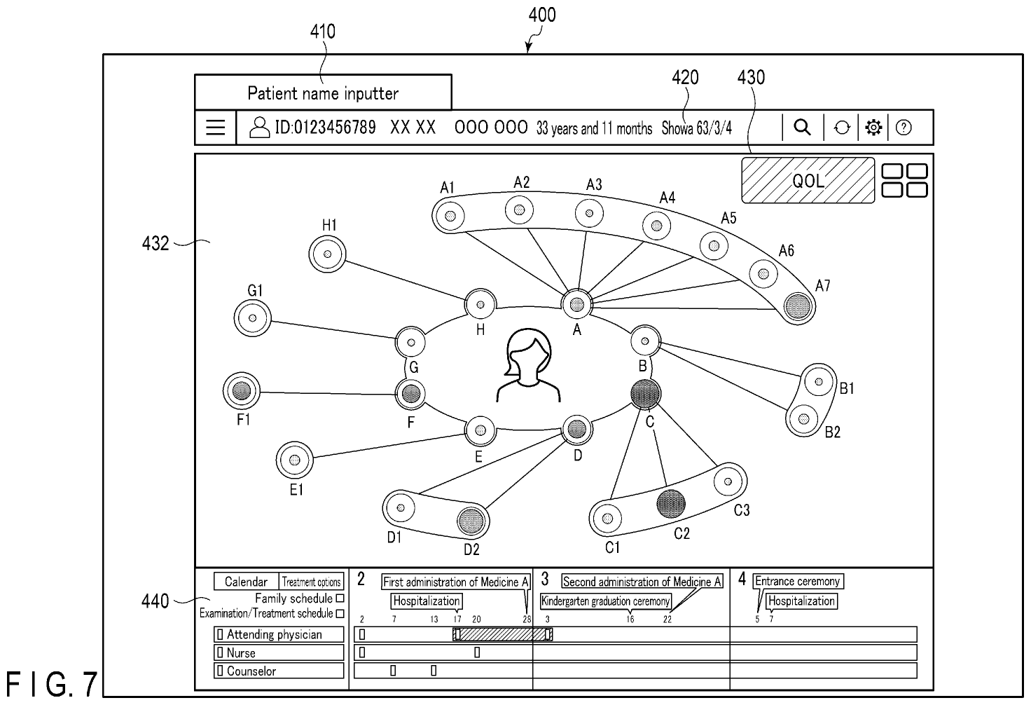

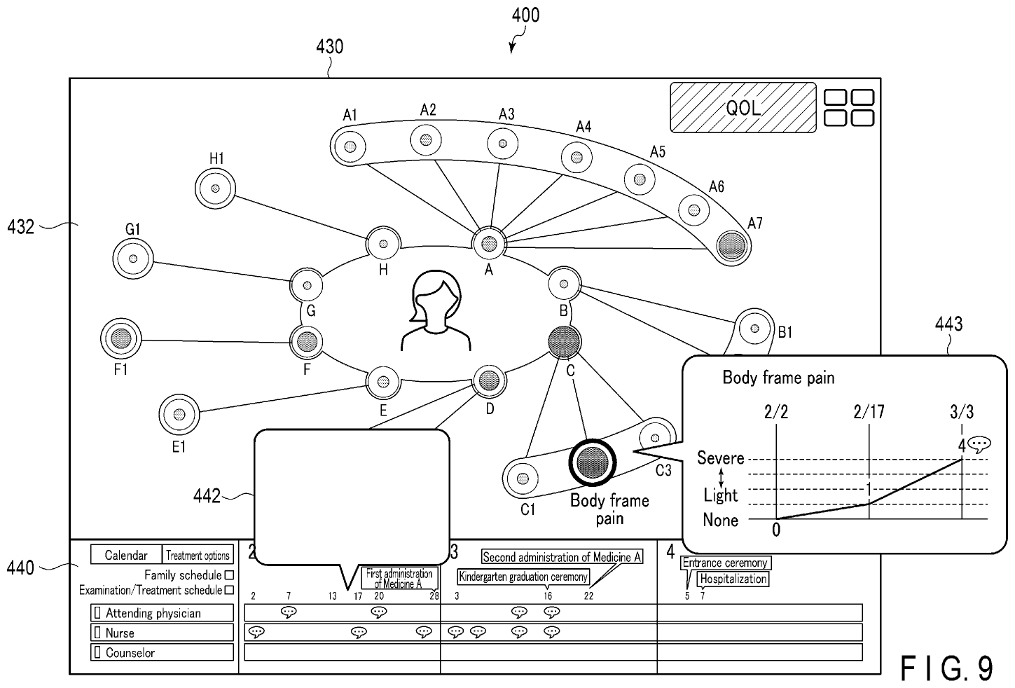

FIG. 9 is a diagram showing an example of the diagnosis support screen in the case in which one of the display items is selected by a user on the diagnosis support screen shown in FIG. 5. As shown in FIG. 7, in response to one of the display items being selected, the related information displayer 440 that displays thereon related information of the selected display item (hereinafter referred to as a selected item) is displayed.

The related information displayer 440 displays thereon the medical record related to the related description associated with the selected item along with the time series information. For example, in the case of the related description being a text, a medical record including the text is displayed. In the case of the related description being a medical record, the medical record is displayed. In response to an icon of a medical record being selected, the contents of the selected medical record are displayed. The related information displayer 440 displays thereon a timeline, and is provided with a display column for each date. A medical record is displayed in the form of an icon in a column of a creation date of the medical record. For example, icons (indexes) of an electronic medical chart created by an attending physician, a nursing record created by a nurse, etc., are displayed on the timeline. Furthermore, the related information displayer 440 is provided with a display column for each of the creators of the medical records. A medical record is displayed in the form of an icon in a column of a creator of the medical record concerned.

Furthermore, the related information displayer 440 displays thereon information on events of a patient. For example, information on a patient's admission, discharge, tests, treatment, and medication is displayed with a corresponding date. A user can infer the cause of matters described in a medical record by checking the context of the relationship between the medical record and an event. For example, in the case where a medical record containing the word “numbness” is displayed, a user can determine whether or not the numbness is caused by drug therapy by grasping whether the creation date of the medical record is before or after the patient's medication date.

Hereinafter, the effects of the diagnosis support system 1 according to the present embodiment will be described.

A conventional diagnosis support system collects mainly information on a disease of a patient (information on a biomedical dimension) and uses this information in making a decision on a treatment option or a treatment plan with a focus on a biomedical dimension (a disease state or a cause). Furthermore, information input by a patient (ePRO: electronic patient reported outcome) has been put to use for the patient to make a decision on a treatment option or a treatment plan. Information input by a patient is taken into an in-hospital system by, for example, a patient himself or herself inputting his or her condition using a smartphone, etc.

On the other hand, the aging of society has involved a major change in the structure of diseases, from acute diseases to chronic diseases such as lifestyle-related diseases. Furthermore, medical progress provides a better prognosis for even a patient with an acute disease, thereby shifting his or her disease to a chronic disease. Under these circumstances, there has been an increasing number of situations in which the disease cannot be cured only by an approach that considers the biomedical dimension based on information on a patient's disease. This increases the importance of medical-care based not only on the biomedical dimension but also on a holistic understanding of a patient gained by carrying out a medical treatment in consideration of the patient's psychological/social dimension while building a trust relationship through communications between the patient and healthcare professional. Furthermore, it is said that social factors account for 50 percent of the health regulating factors.

Herein, information on the patient's psychological/social dimension may be stored in a hospital information system such as an electronic health chart, etc. The information on the patient's psychological/social dimension is entered into a medical record such as an electronic medical chart, a nursing chart, a cancer consultation record, etc., and entry items are often limited. In addition, as integrated information for a clear multi-views of a patient, displaying such a medical record and information input by the patient all together is not performed.

Furthermore, various items of information such as information on the biomedical dimension and information on the psychological/social dimension obtained during medical interviews, medical treatments, and consultations with healthcare professional are stored in each of the electronic medical chart, the nursing record, the consultation record, etc.; however, they are not recorded in a form that facilitates use in medical treatment carried out based on holistic understanding of the patient. Further, simply collecting and presenting the information obtained by various healthcare professional merely results in an increased information amount, and it is difficult to utilize the collected information in the busy daily practice of medical treatment. For example, contents of a recorded chart cannot be considered simply by presenting the amount of the recorded charts, and thus no holistic understanding of a patient can be provided.

These circumstances bring about a demand for a support system that collects and integrates information obtained by various healthcare professionals interacting with a patient from various dimensions, and supports a medical treatment using the collected information on various dimensions of the patient.

To address the above problem, the diagnosis support system 1 according to the present embodiment acquires medical data including medical records acquired at a plurality of points in time and performs analysis processing on the medical data. The analysis processing is, for example, natural language processing. Thereafter, the diagnosis support system 1 extracts descriptions related to biomedical items and descriptions related to psychosocial items from medical data based on a result of the analysis processing for each of the display items, and calculates the information amount of the biomedical items and amount of the psychosocial items using a result of the extraction. The diagnosis support system 1 causes the biomedical items and psychosocial items to be displayed in a mode according to the information amount. The information amount is calculated using, for example, the number of descriptions extracted as those related to the biomedical or psychosocial items or the similarity between the plurality of extracted descriptions. Descriptions are used, for example, in unit of a medical record, in unit of a text included in the medical record, in unit of a paragraph included in the medical record.

The above configuration enables the diagnosis support system 1 according to the present embodiment to cause the diagnosis support screen to display thereon a multi-views of a patient, including not only the biomedical information on a disease such as clinical information but also the psychosocial information such as his or her intentions, his or her sense of values, etc., obtained by a plurality of healthcare professionals.

For example, in response to a cancer patient's complaint of “numbness”, supportive care is given because the numbness is highly likely to be caused by a side effect of a drug in the case where the timing at which the numbness occurred is after the start of drug therapy. On the other hand, in the case where the timing at which the numbness occurred is not relevant to the start of drug therapy, additional tests are performed because the numbness may be caused by bone metastasis. As described above, the next action can be effectively determined by using a cancer patient's complaint of “numbness” as information. However, information on numbness and pain is based on information that a patient voluntarily states, and is not available from all patients. Furthermore, in the case where numbness or pain is mentioned in a conversation between a healthcare professional and a patient, the conversion is recorded not only in an electronic medical chart but also in a nursing record or consultation record. The diagnosis support system 1 according to the present embodiment enables sentences describing for psychosocial information that have not been sufficiently used in the past to also be treated as related information by making all sentences in a description subject to natural language processing. This allows a healthcare professional to make full use of information that can assist an inference lying somewhere in a description.

Furthermore, this allows healthcare professionals or an interprofessional work team to check information collected by healthcare professionals through interactions with patients while examining the information from the viewpoints of their respective specialties, and to pick up (screening or triage) those who are in need or should be given an intervention. In addition, this allows efficient access to information necessary for understanding a patient and considering the next action, such as what is important to the patient, why some change in the patient has occurred, what kind of care and treatment should be done, etc. In other words, in the case of integrating and aggregating biopsychosocial information obtained by various medical professionals and considering treatment options without increasing the burden due to an increase in the information amount, medical-care based on biopsychosocial models (holistic medicine, patient-centered care) that use psychosocial information such as a patient's view and preference and sense of value can be supported in the field of medical examinations, communications with patients, or communications between healthcare professionals.

Furthermore, the diagnosis support system 1 according to the present embodiment enables items constituting a multi-views of a patient to be displayed in a mode according to the corresponding information amount. For example, by changing an icon or a font size of a psychosocial item in accordance with the information amount, the information amount of each item can be visualized and displayed. An item with the large information amount can be regarded as an important item in diagnosis. Therefore, a user can easily grasp an item to be confirmed by grasping the information amount.

Furthermore, the diagnosis support screen can display thereon a description related to the selected item along with the time series information. For example, by displaying a description in such a manner that a creation date of the description can be grasped, a user can estimate based on the creation data a factor that caused the change in a patient. In addition, by further displaying event information such as a hospitalization date, a discharge date, and a medication date, a user can accurately estimate a factor that caused the change in the patient. Furthermore, information on a creator of the medical record may also be displayed. As described above, in the present embodiment also, patient information collected and integrated from various viewpoints can be shared among the medical team and used for decision-making.

### Modifications of First Embodiment

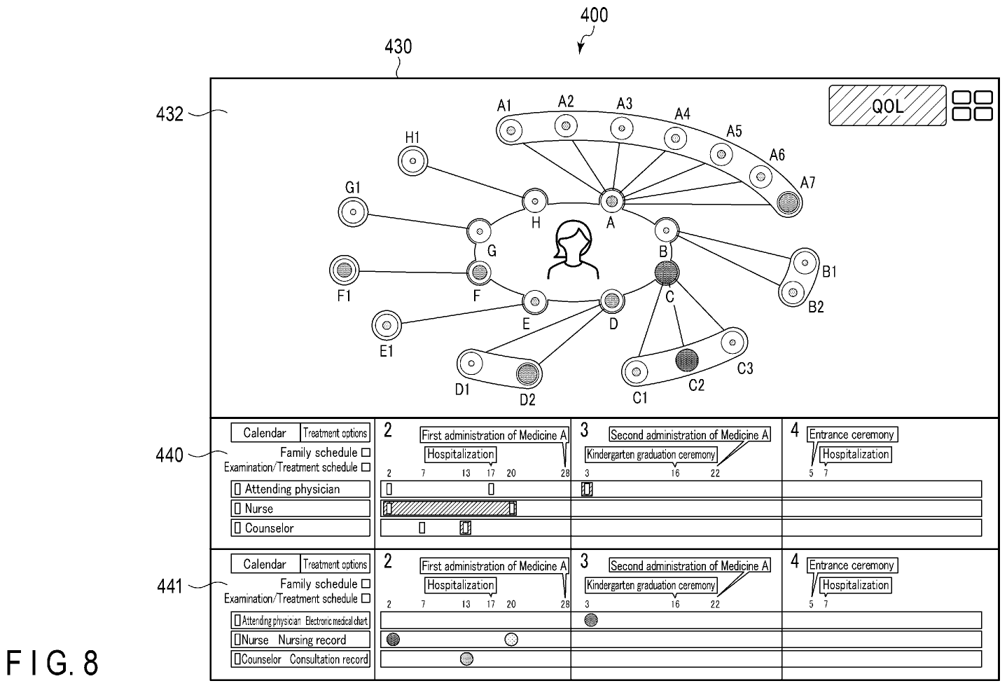

The information amount may be calculated using weighting according to the strength of the relationship between a display item and a description. For example, weighting may be performed in such a manner that a description having a higher degree of similarity to a display item has the larger information amount, and a total value of information amount of the description may be calculated as the information amount. In such a case, a related information displayer 441 that displays thereon a weight of each description may be displayed, as shown in FIG. 8. In the example shown in FIG. 8, the related information displayer 441 is displayed in such manner that the color of a display icon of a description becomes darker as the degree of similarity to the selected item increases. The related information displayer 441 may display thereon an icon indicating the information amount of each description. Furthermore, both of the similarity and the information amount may be indicated using a single display icon. For example, the similarity may be indicated according to the color, and the information amount may be indicated according to the size of the display icon, or the information amount may be indicated according to the color of the display icon, and the similarity may be indicated according to the size of the display icon.

Furthermore, as shown in FIG. 9, in response to one of the descriptions displayed in the related information displayer 440 being selected, a content displayer 442 indicating the contents described in the selected description may be displayed. The content displayer 442 may display thereon, for example, text in the description either as it is or in a summary. Furthermore, in the case where the selected description is video data, a video may be played on the content displayer 442, or keyframes of the video may be displayed thereon. In the case where the selected description is voice data, voice data is played instead of the content displayer 442 being displayed.

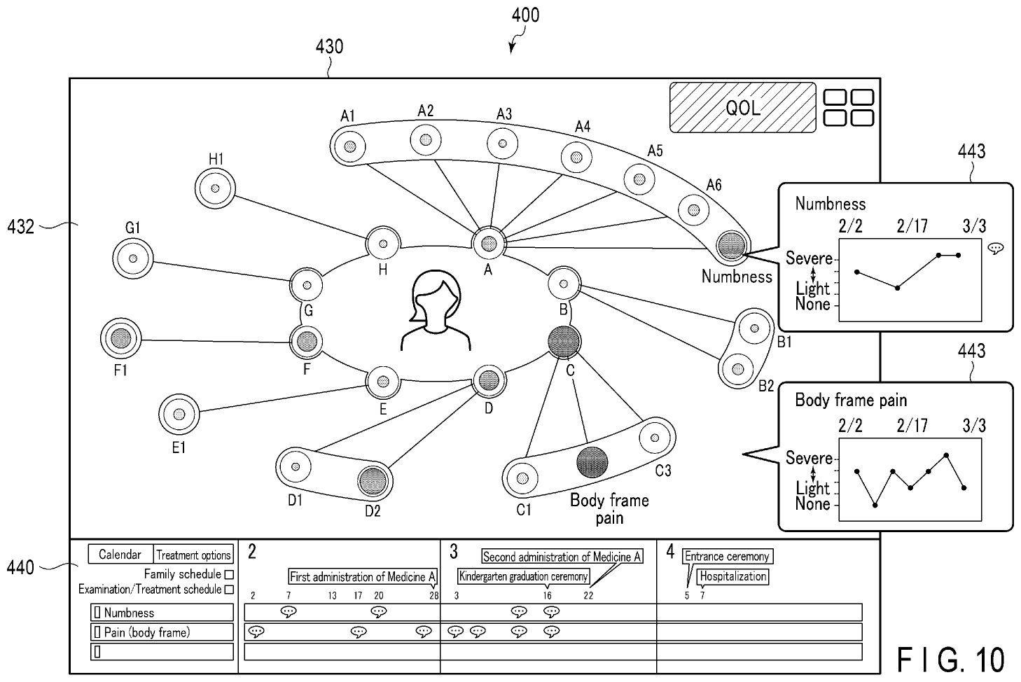

Furthermore, as shown in FIG. 9, in response to one of the display items being selected, a temporal change displayer 443 indicating a change over time of a parameter related to the selected item may be displayed. In such a case, for each display item, parameters related to a corresponding display item are digitized and stored in advance using a linguistic analysis result of each description associated with the corresponding display item. Thereafter, in response to one of the display items being selected, the display controller 350 reads a parameter of the selected display item and causes the temporal change displayer 443 to display thereon a graph indicating a change over time of the parameter related to the display item. A user can easily grasp the change over time of the parameter related to the item that he or she wishes to confirm. Furthermore, as shown in FIG. 10, the temporal change displayer 443 may display graphs for the plurality of display items simultaneously. The user can compare the plurality of items that he or she wishes to confirm. In such a case, the related information displayer 440 is provided with a display column for each of the selected descriptions, and an associated description is displayed in a column of the corresponding creation date.

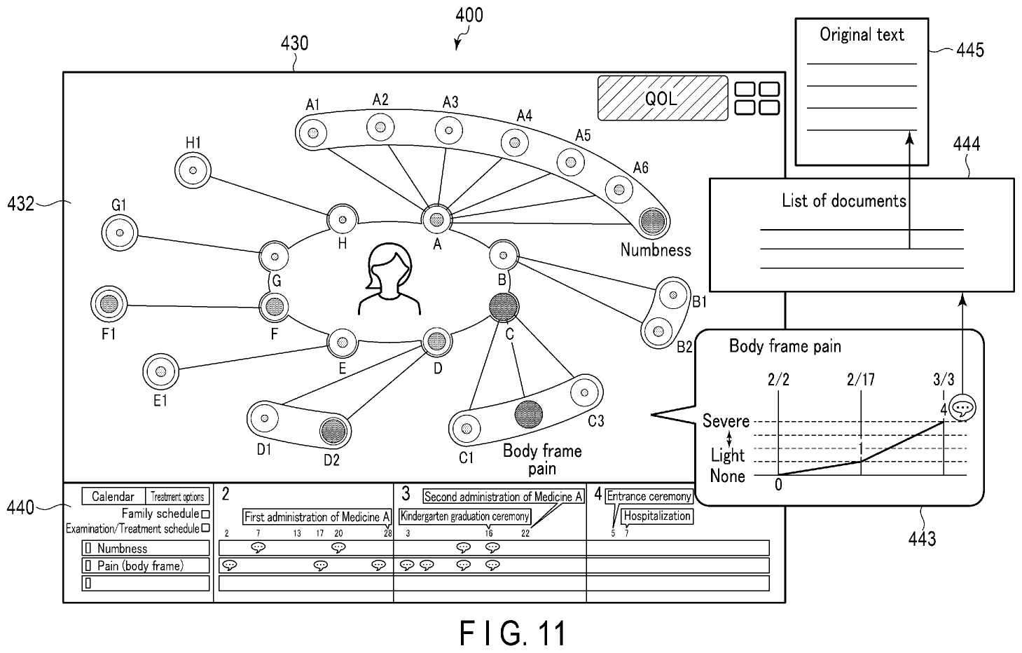

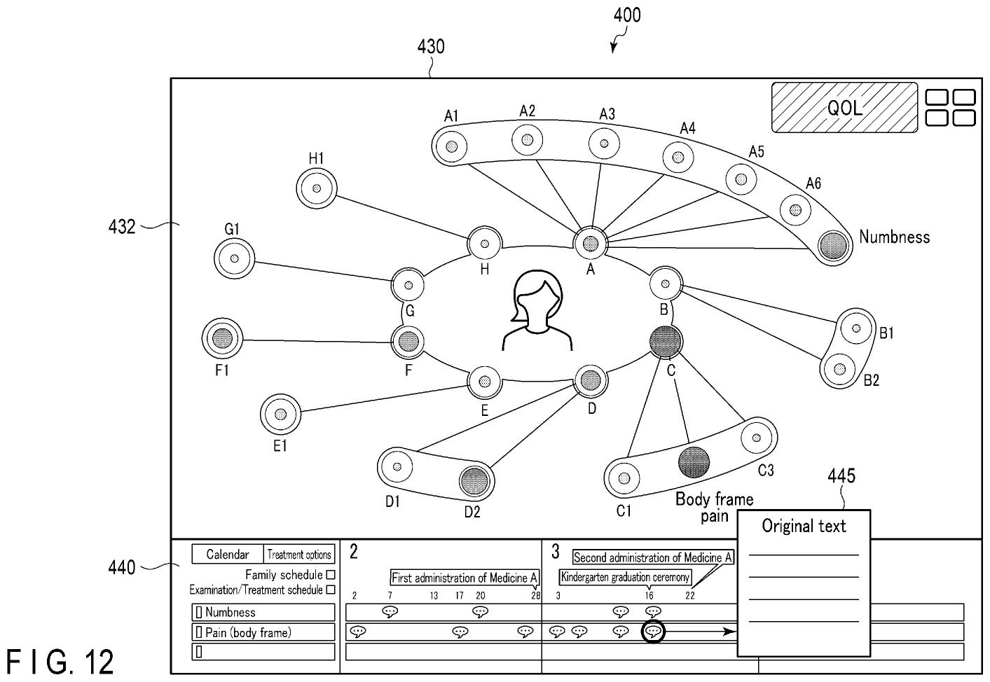

Furthermore, as shown in FIG. 11, in response to an icon displayed on the temporal change displayer 443 being selected, a list displayer 444 that displays thereon a list of descriptions associated with the selected item may be displayed. The list displayer 444 may display thereon, for example, a part of the text or a summary of each medical record associated with the selected item. Furthermore, in response to one of the medical records displayed in the list displayer 444 being selected, an original text displayer 445 that displays thereon original text of the selected medical record may be displayed. The original text displayer 445 displays thereon, for example, the entire text of the medical record without any change. A user can easily confirm a list of related documents. As shown in FIG. 12, in response to a medical record displayed on the temporal change displayer 443 being selected, the original text displayer 445 that displays thereon original text of the selected medical record may be displayed.

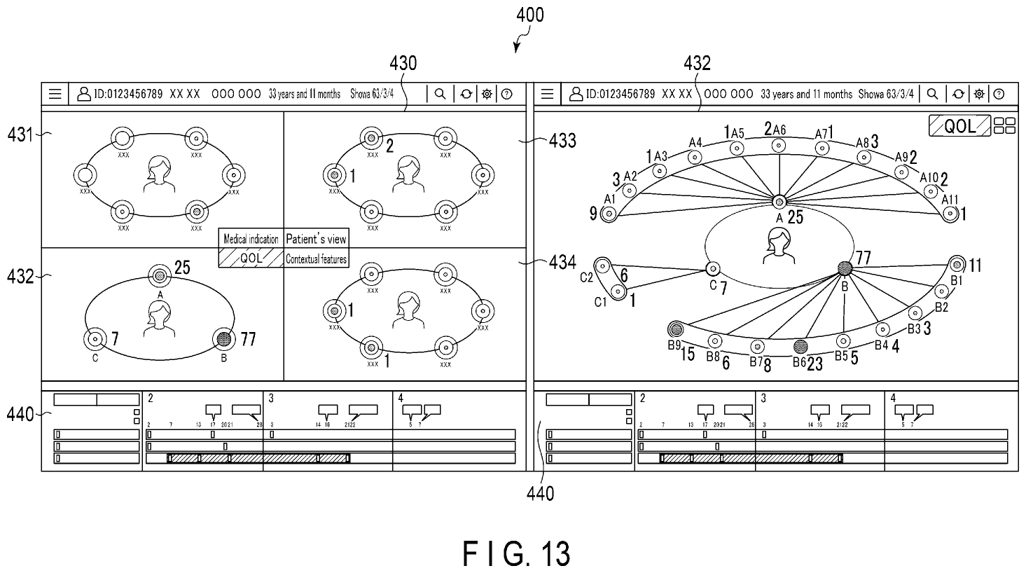

Furthermore, as shown in FIG. 13, both the major item display screen that displays thereon display items for each major item and a detailed item display screen that displays thereon display items classified into selected major items may be displayed.

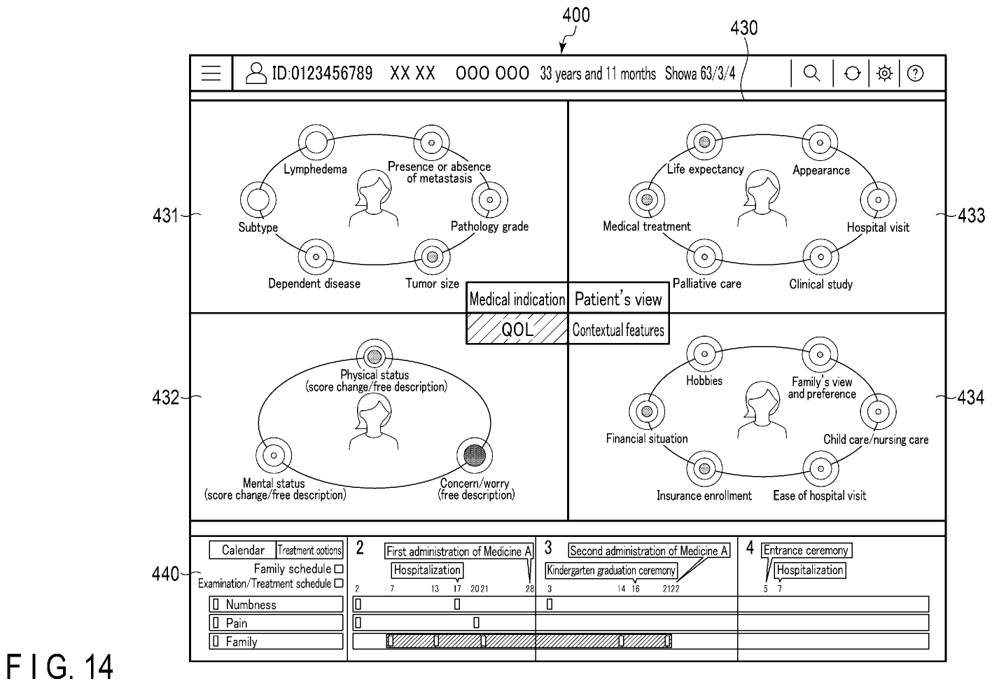

Furthermore, as shown in FIG. 14, even if the display item is not selected, the related information displayer 440 may be displayed. In such a case, one or more display items are set in advance, and the medical record associated with the set item is displayed on the related information displayer 440.

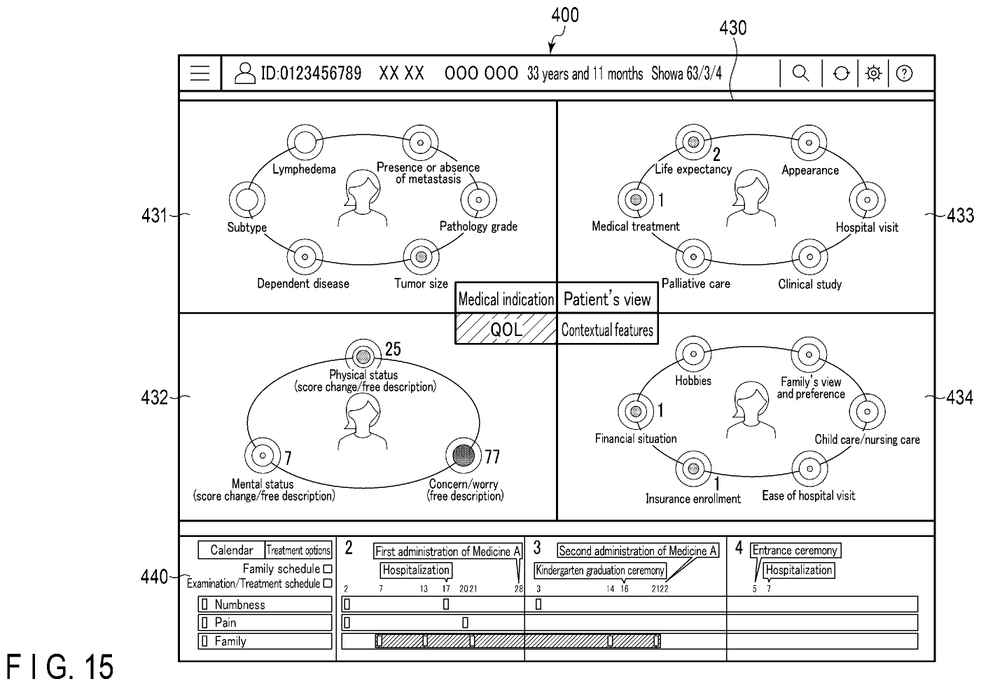

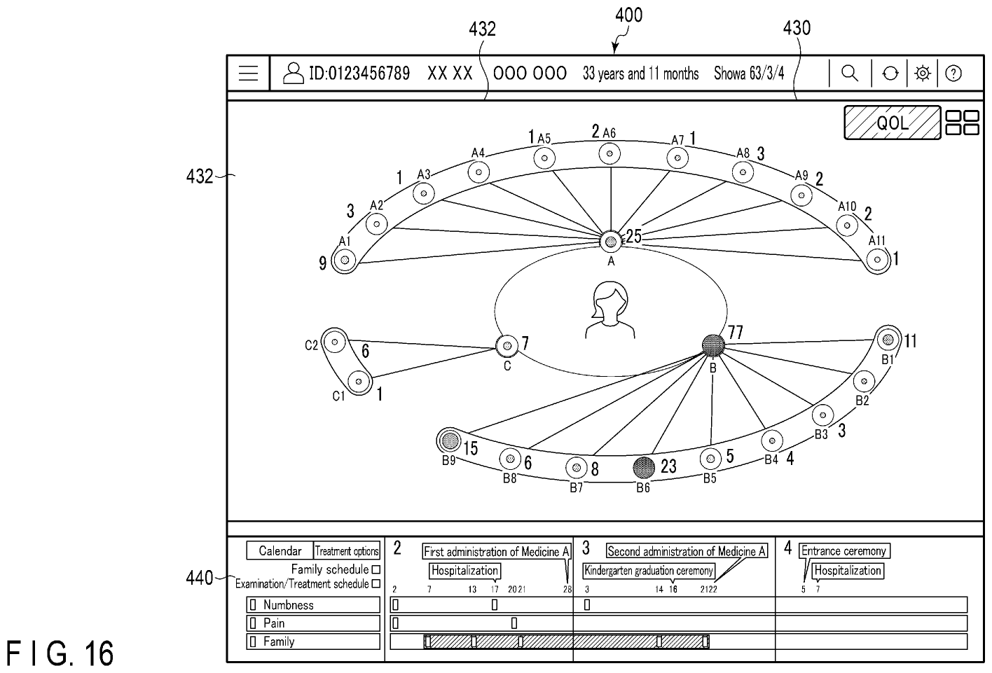

Furthermore, as shown in FIG. 15 and FIG. 16, a numerical value of the information amount may be displayed along with a name of the display item. A user can grasp the information amount in more detail by confirming a quantified score (information amount) as well as the size of an icon.

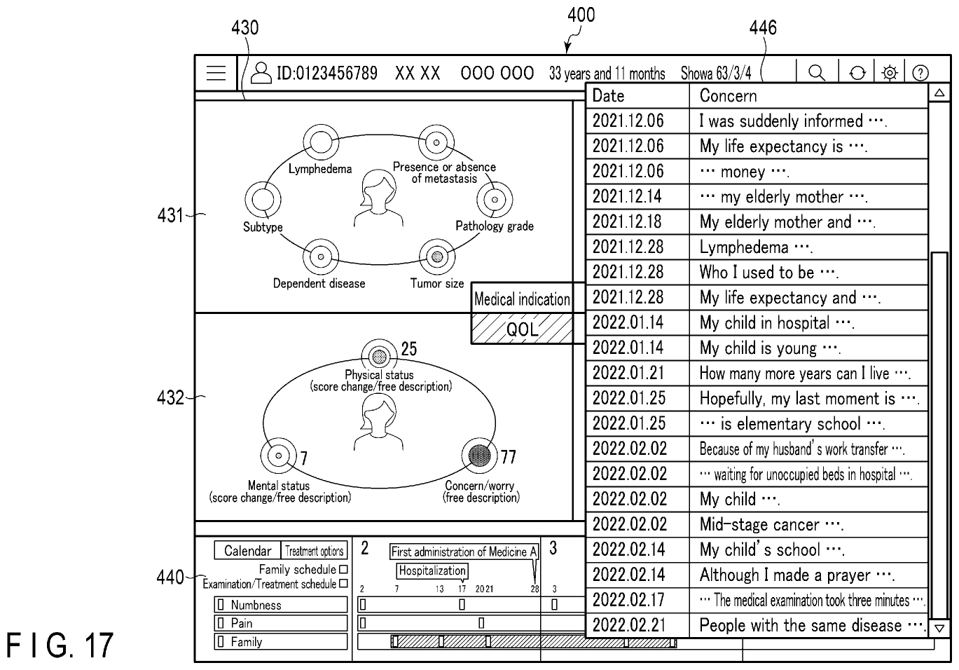

Furthermore, as shown in FIG. 17, a full-text displayer 446 that displays thereon all descriptions associated with each display item may be displayed. The full-text displayer 446 displays thereon, for example, creation dates of medical records each including a related description and the full text of their contents in order of creation date.

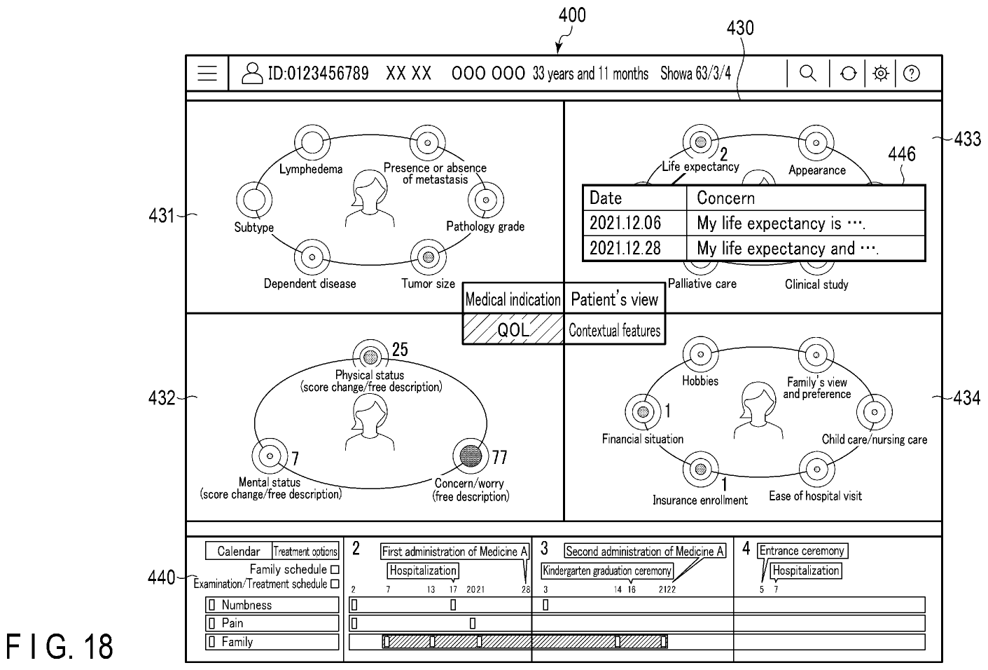

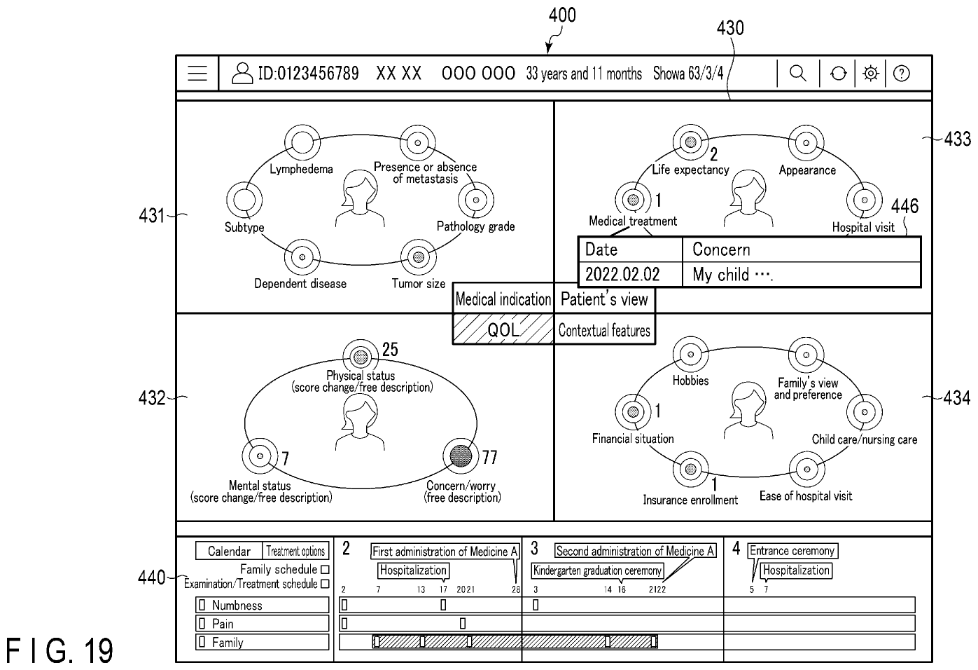

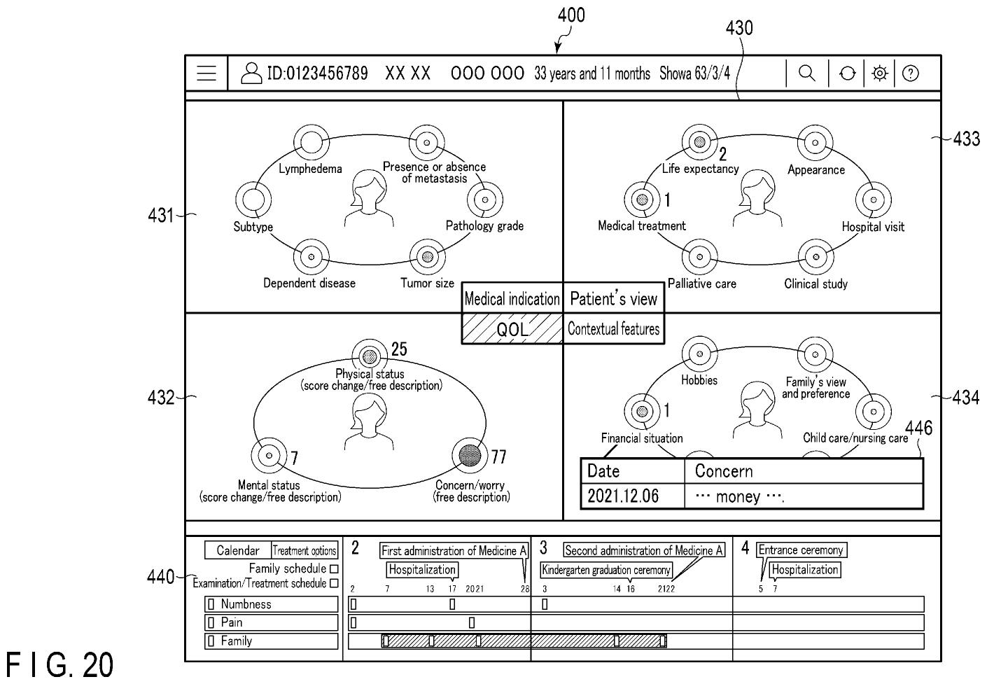

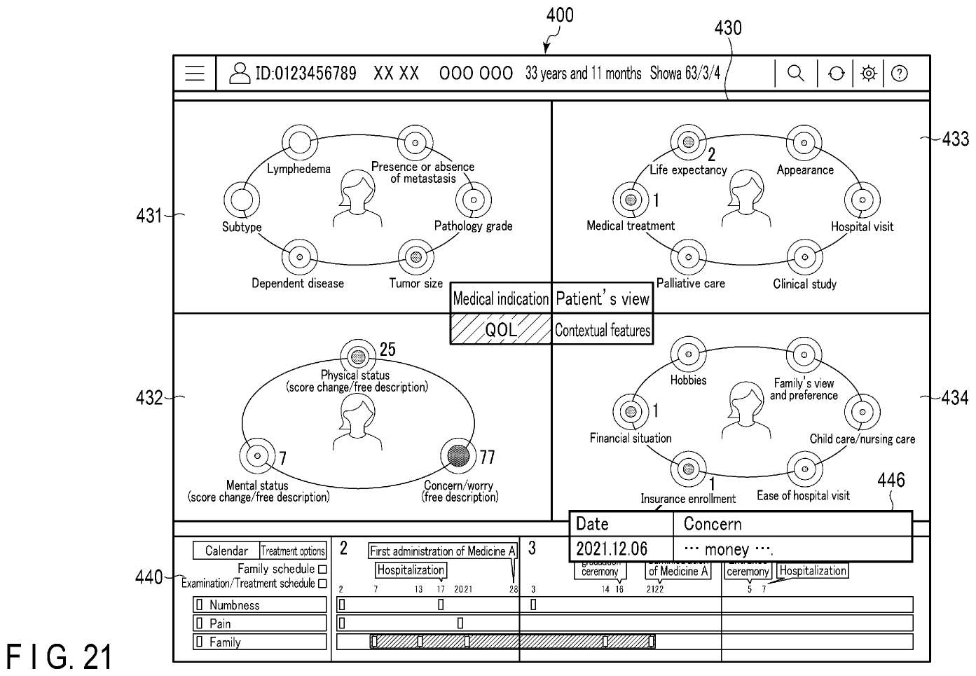

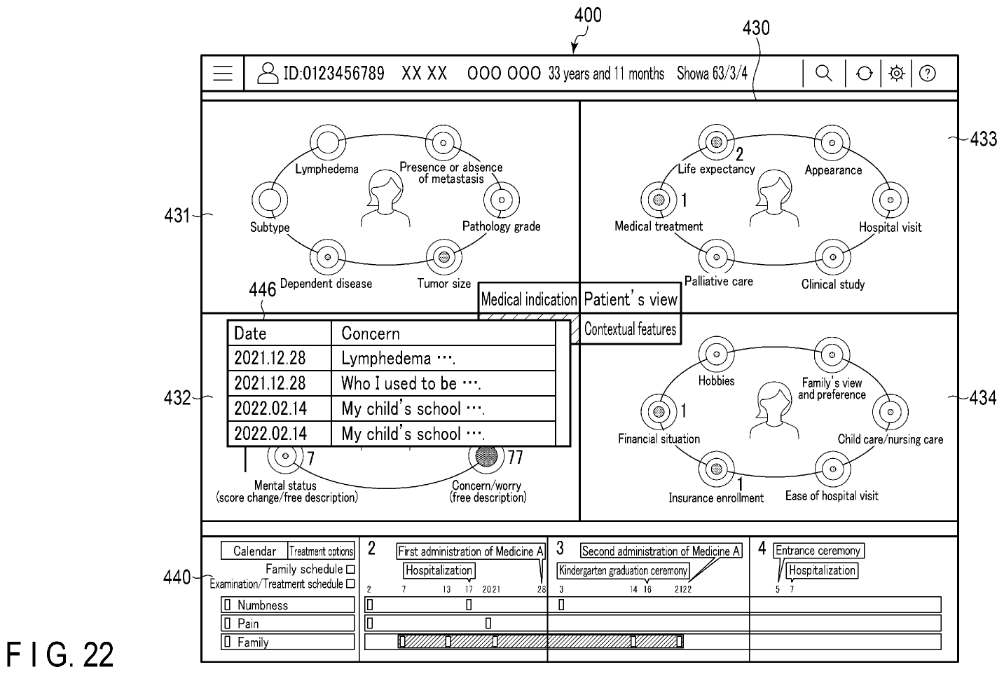

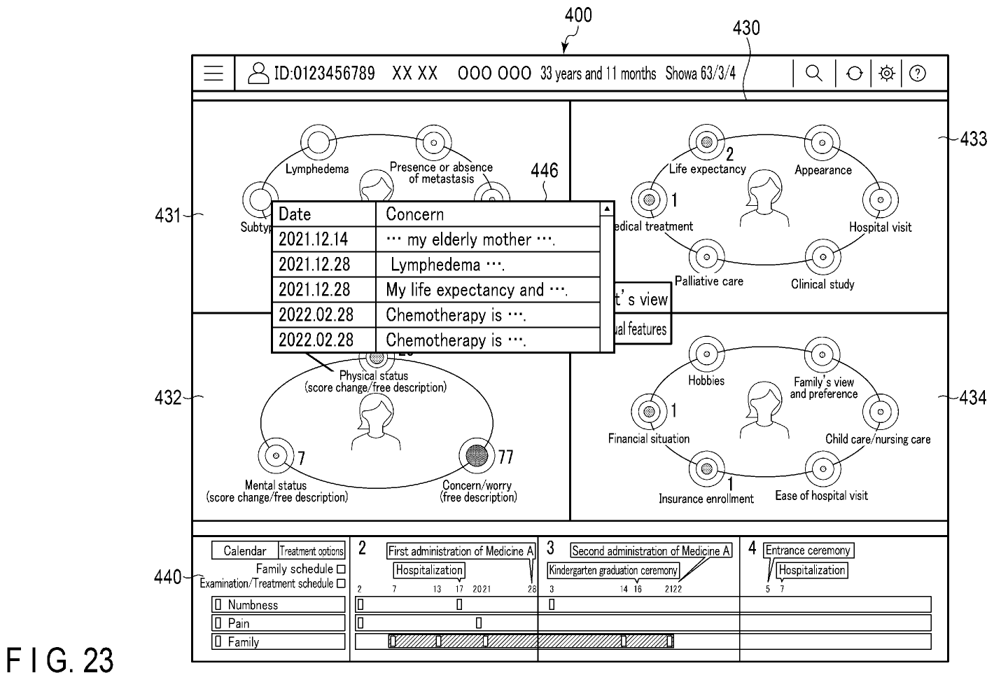

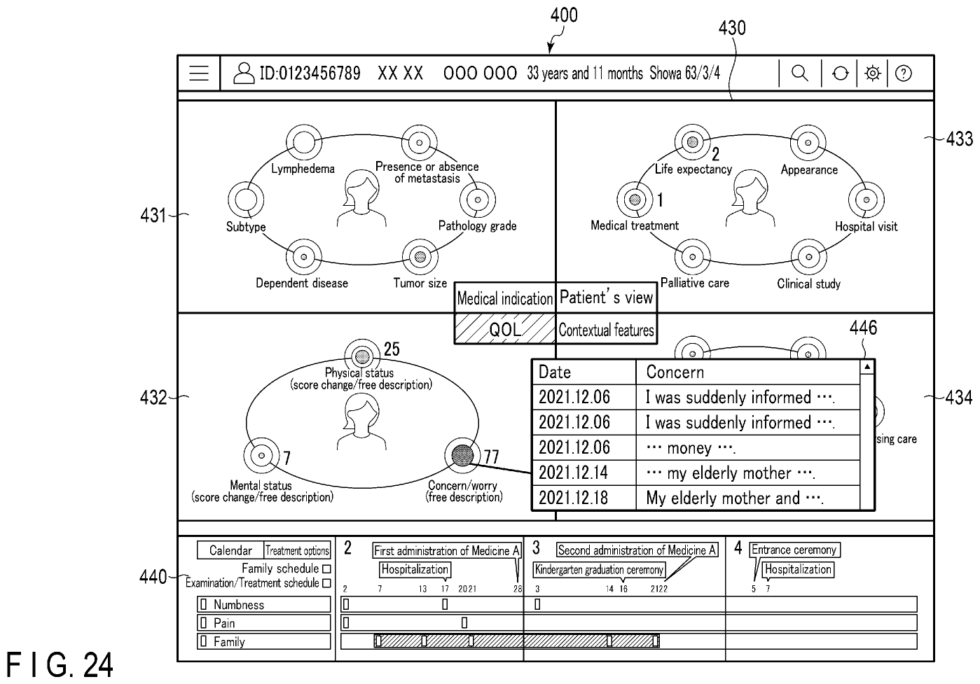

Furthermore, as shown in FIG. 18, in the case where one of the display items is selected on the major item display screen of the information displayer 430, the full-text displayer 446 that displays thereon all descriptions associated with the selected item may be displayed. FIG. 18 shows an example of the case in which the number of associated descriptions is used as the information amount. As shown in FIG. 18, in the case where the item of “life expectancy” with which two descriptions are associated is selected, two descriptions are displayed on the full-text displayer 446. Furthermore, as shown in FIG. 19 to FIG. 21, in the case where the item of “treatment”, “economic situation” or “subscription to insurance” with which one description is selected is selected, one description is displayed on the full-text displayer 446. Furthermore, as shown in FIG. 22 to FIG. 24, in the case where an item with a large information amount such as “mental status”, “physical status”, “concern/worry”, etc., is selected, a portion of the associated descriptions is displayed on the full-text displayer 446, and it may be displayed in such a manner that all of the descriptions can be confirmed by scrolling the full-text displayer 446.

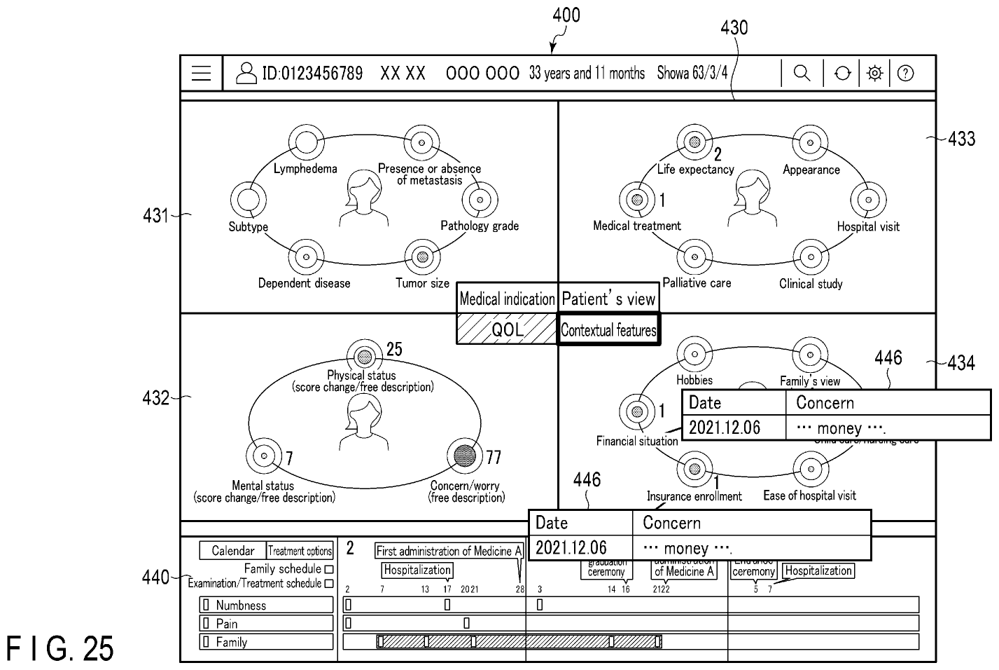

Furthermore, as shown in FIG. 25, in the case where one of the major items is selected on the major item display screen of the information displayer 430, the full-text displayer 446 may be displayed for all of the display items included in the selected major item.

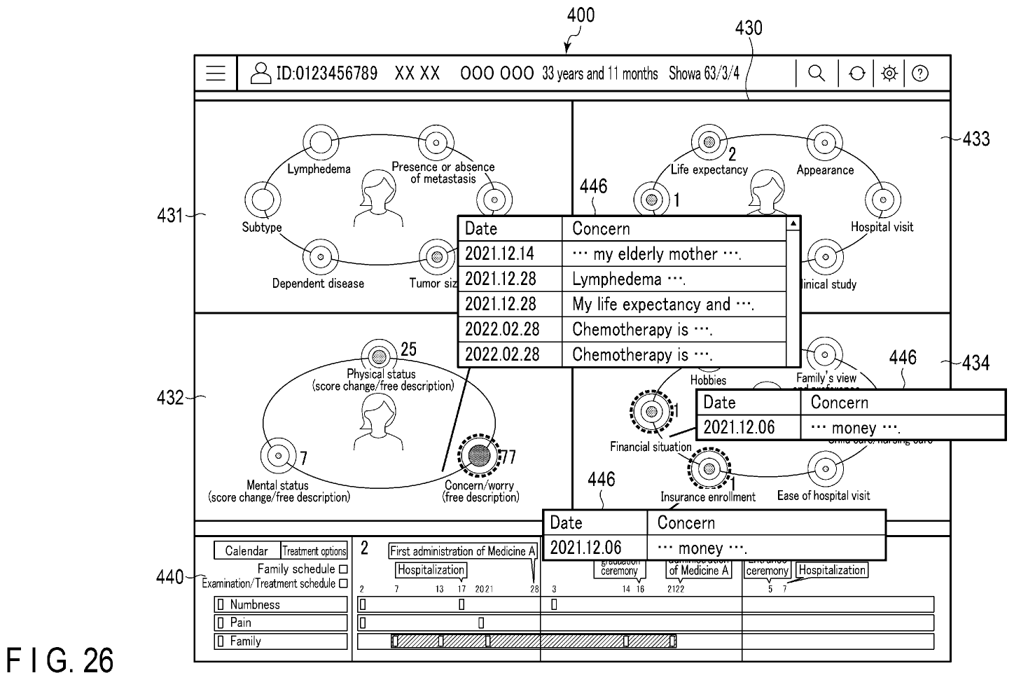

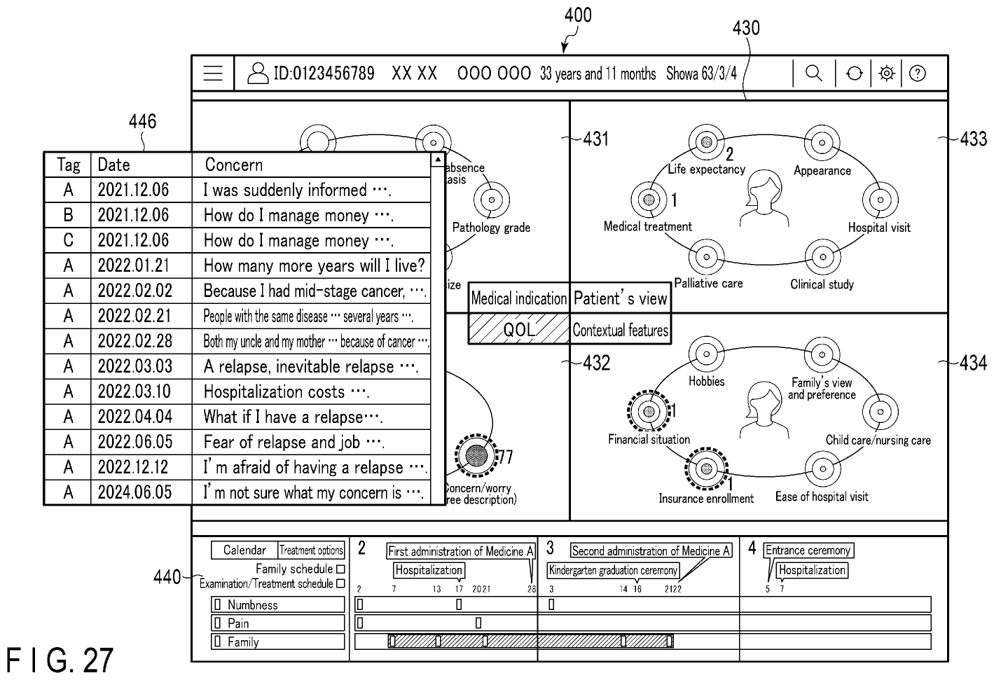

Furthermore, as shown in FIG. 26, in the case where a plurality of display items is selected on the major item display screen of the information displayer 430, the plurality of full-text displayers 446 corresponding to each selected display item may be displayed simultaneously. Furthermore, as shown in FIG. 27, in the case where the plurality of display items is selected, all of the descriptions associated with each selected display item may be displayed on one full-text displayer 446.

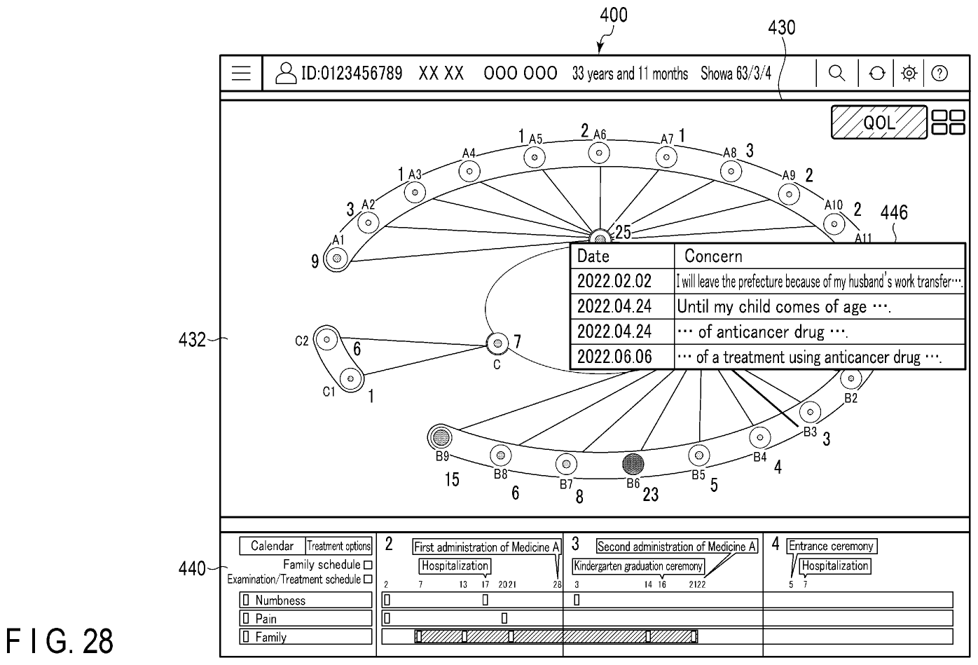

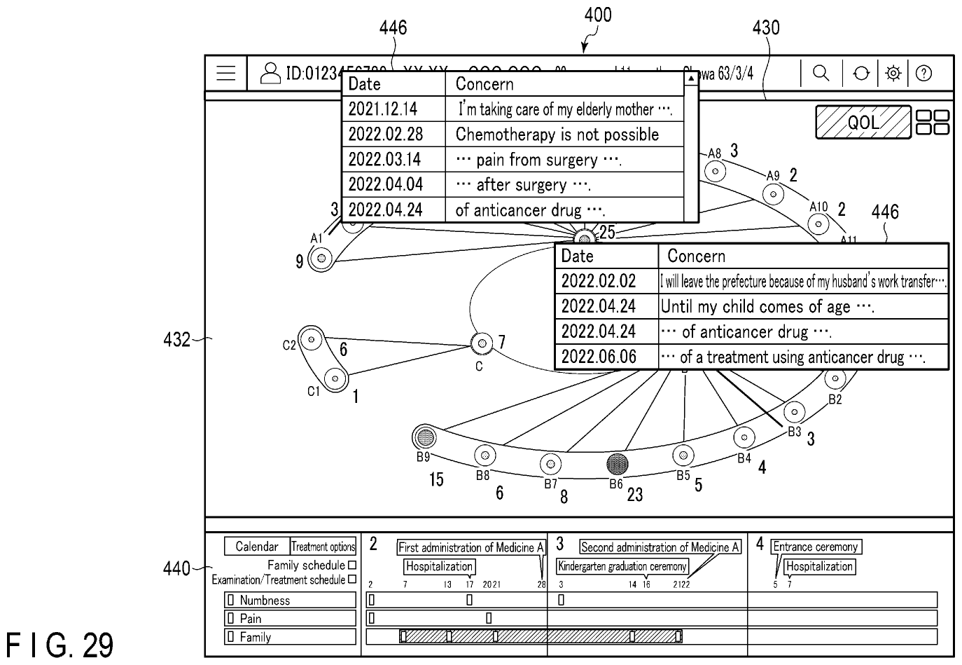

Furthermore, as shown in FIG. 28, in the case where one of the display items is selected on the detailed item display screen of the information displayer 430, the full-text displayer 446 may be displayed in a similar manner to that shown in FIG. 18. Furthermore, as shown in FIG. 29, in the case where the plurality of display items is selected on the detailed item display screen of the information displayer 430, the plurality of full-text displayers 446 corresponding to each selected display item may be displayed simultaneously in a similar manner to that shown in FIG. 26.

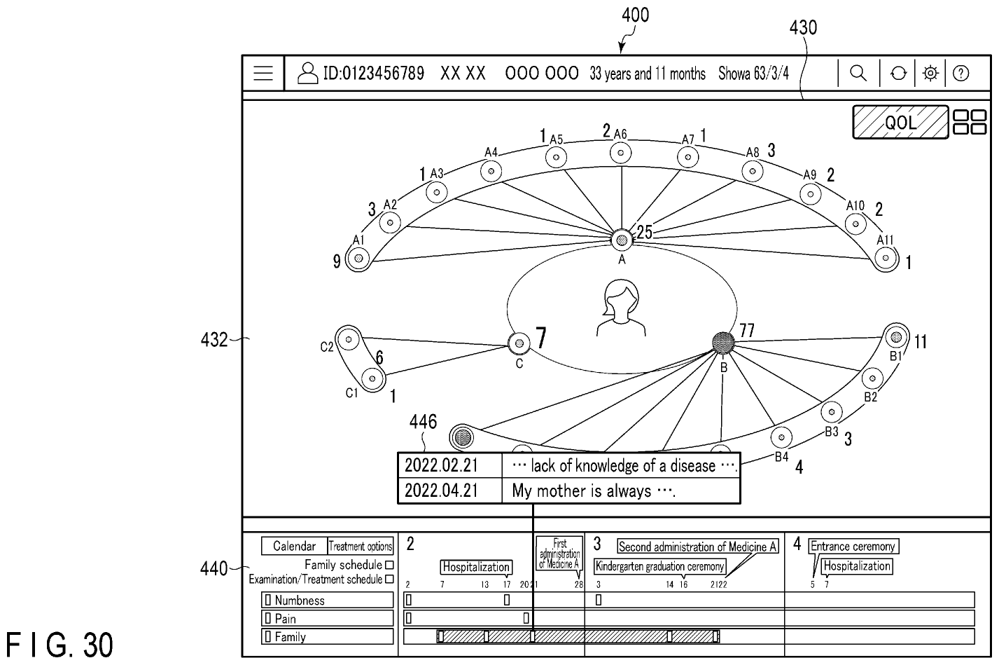

Furthermore, as shown in FIG. 30, in the case where one of the dates is selected on the related information displayer 440, the full-text displayer 446 that displays thereon all descriptions created on the selected date from among the descriptions associated with the selected item may be displayed.

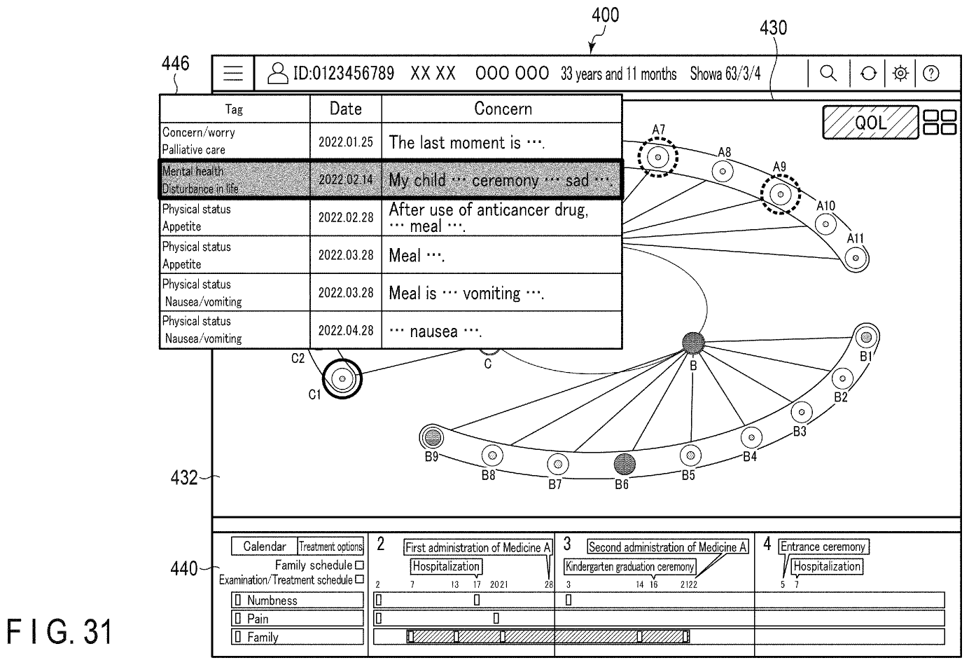

Furthermore, as described in FIG. 27, in the case where all descriptions associated with the plurality of selected display items are displayed on one full-text displayer 446, as shown in FIG. 31, in addition to the creation date of the description and the full text of its contents, a tag indicating a name of the corresponding display item may be displayed on the full-text displayer 446. Yet further, as shown in FIG. 31, in the full text of the contents, a term related to the display item may be emphasized and displayed. For example, in the example shown in FIG. 31, a term related to the display item is displayed in bold.

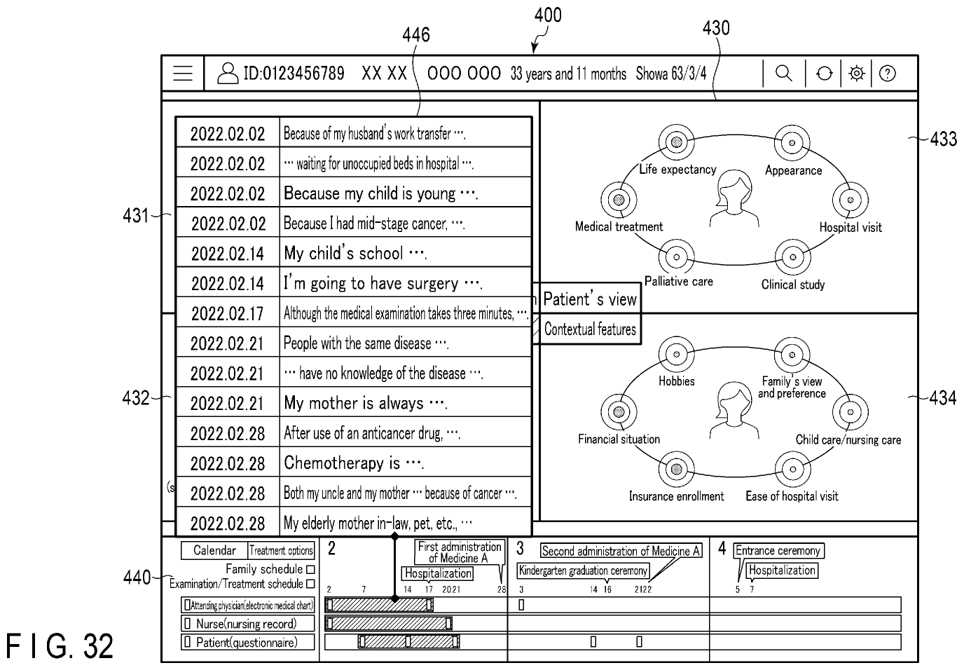

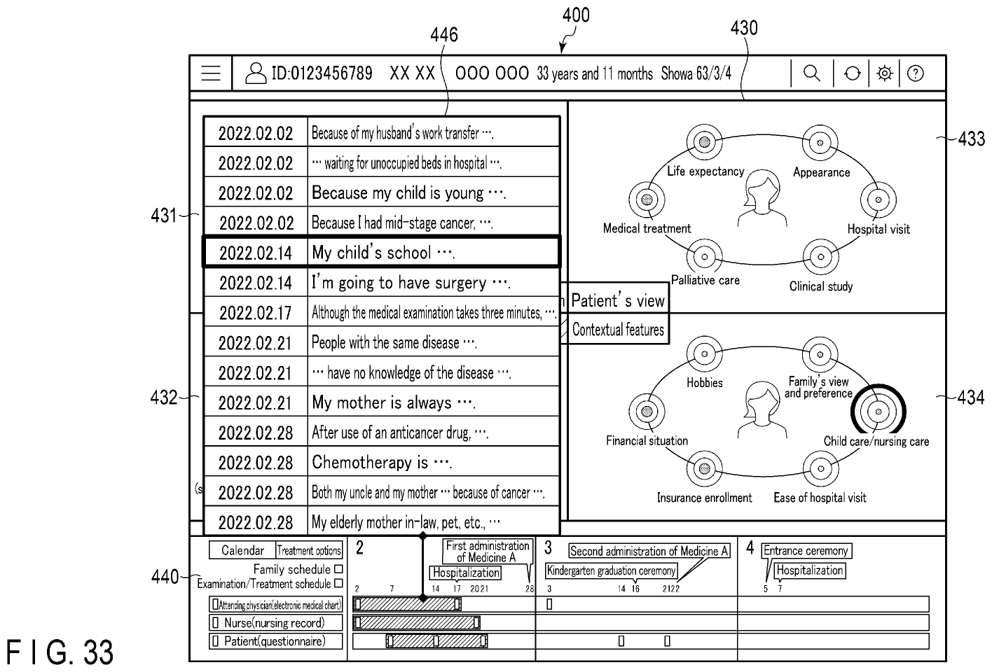

Furthermore, as shown in FIG. 32, in the case where a specific period is selected on the related information displayer 440, the full-text displayer 446 that displays all descriptions created during the aforementioned period from among the descriptions associated with the selected item may be displayed. Furthermore, in the case where one of the medical items displayed on the related information displayer 440 is selected while the full-text displayer 446 shown in FIG. 32 is displayed, as shown in FIG. 33, the description corresponding to the selected medical item from among the descriptions displayed on the full-text displayer 446 may be emphasized and displayed. For example, in the example shown in FIG. 33, a description corresponding to the selected medical item is surrounded by a thick frame and is displayed.

### Second Embodiment

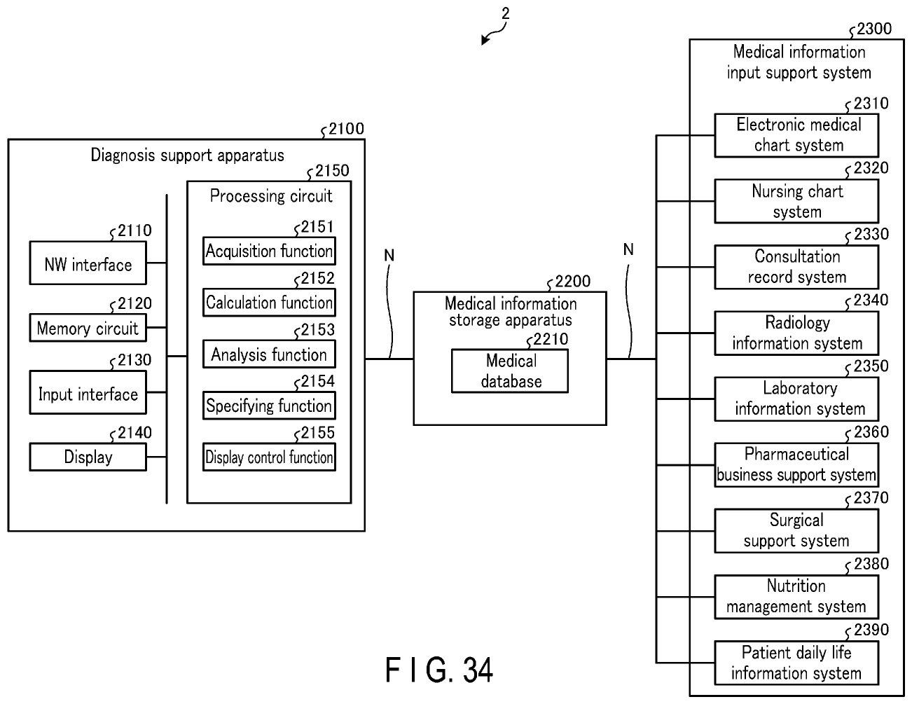

The overall configuration of the diagnosis support system according to the second embodiment will be described. FIG. 34 is a block diagram showing an example of the configuration of the diagnosis support system 2 according to the second embodiment. The diagnosis support system 2 includes a diagnosis support apparatus 2100, a medical information storage apparatus 2200, and a medical information input support system 2300. The diagnosis support system 2 may be referred to as a clinical decision support system.

The diagnosis support apparatus 2100, the medical information storage apparatus 2200, and the medical information input support system 2300 are connected, for example, via a network N. The network N can be realized in any form regardless of whether it is wired or wireless as long as it enables communication between the diagnosis support apparatus 2100, the medical information storage apparatus 2200, and the medical information input support system 2300.

The diagnosis support apparatus 2100 is an apparatus that presents information on a subject collected from various viewpoints to a user such as a doctor, nurse, or counselor together with a feature amount (an accumulated value or a change value (hereinafter also referred to as a “fluctuation”) related to a feature). Examples of the information indicating a “fluctuation” include a fluctuation value. The fluctuation value is an example of the feature. The configuration of the diagnosis support apparatus 2100 will be described later. The diagnosis support apparatus 2100 may be referred to as a clinical decision support apparatus.

The medical information storage apparatus 2200 stores a medical database 2210 related to medical information. The medical information storage apparatus 2200 is incorporated into, for example, a Hospital Information System (hereinafter referred to as an “HIS”). The medical database 2210 may be referred to as a medical information DB.

The medical database 2210 stores information on a plurality of subjects. Examples of the subjects include a patient. For example, the medical database 2210 stores patient information, medical information, and personal information.

Examples of the patient information include information in the electronic medical chart related to a subject (for example, a patient ID, a name, date of birth, information on a disease, weight, height, etc.).

Examples of the medical information include insurance points, a drug price, and a standard monthly remuneration according to the treatment method for the disease, various items of information on the subject entered by a healthcare professional (including a cancer counselor, etc.) such as a doctor, a nurse, etc., information recording dialogue between the subject and a healthcare professional such as a doctor, and information on a result of natural language processing (hereinafter also referred to as a “language processing result”) on information recording, e.g., a dialogue between the subject and the medical stuff. The medical information may be referred to as a description. The medical information is an example of the medical data.

Herein, a target of the natural language processing may be, for example, text data of, e.g., results of various searches by a subject in addition to information described in a description and voice data from a dialogue between the subject and a doctor, a nurse, a counselor, etc. Examples of the text data may include text data indicating search results, search history, etc., from a subject's search for his or her own disease on the Internet, etc. In the case where a target of the natural language processing is voice data, a language processing result corresponds to an analysis result of voice.

Voice data or text data are input, for example, via an information processing apparatus, etc., which constitutes the medical information input support system 2300. The language processing result is stored in the form of Frequently Asked Questions (FAQ) into the medical database 2210.

Examples of the personal information include information on a -specific event (i.e., an event unique to the subject), schedule information (action schedule) of the subject or the subject's family, monthly remuneration (economic information) of the subject or a household to which the subject belongs, private insurance in which the subject is enrolled and the details of the private insurance, information indicating the subject's preferences, information indicating the subject's values (for example, matters considered important in daily life, matters considered important for treatment of a disease), information described in an interview sheet, a questionnaire, etc., a result of the language processing on text data of various search results by the subject, etc.

Hereinafter, patient information, medical information, and personal information stored in the medical database 2210 are collectively referred to as medical information.

The medical information input support system 2300 is a system that collects medical information and registers the collected medical information in the medical database 2210. The medical information input support system 2300 includes the electronic medical chart system 2310, the nursing chart system 2320, the consultation record system 2330, the radiology information system 2340, the laboratory information system 2350, the pharmaceutical service support system 2360, the nutrition management system 2380, and the patient daily life information system 2390.

The configuration of the medical information input support system 2300 shown in FIG. 34 is an example, and the medical information input support system 2300 may not have all pieces of the above configuration. Furthermore, the medical information input support system 2300 may have a configuration other than the above configuration.

The electronic medical chart system 2310 is an information system that manages an electronic medical chart for recording medical details. The electronic medical chart system 2310 includes, for example, an electronic medical chart server apparatus and an electronic medical chart terminal apparatus.

The electronic medical chart server apparatus is a computer apparatus that performs processing related to the management of electronic medical charts. The electronic medical chart server apparatus is composed of one or more computer apparatuses, depending on the scale of the system. The electronic medical chart terminal apparatus is used by a doctor, a nurse, etc., who inputs and refers to electronic medical charts. One or more electronic medical chart terminal apparatuses are provided, depending on the scale of the system. The electronic medical chart server apparatus and the electronic medical chart terminal apparatus are connected to the network N.

Information input via the electronic medical chart terminal apparatus by a doctor, a nurse, etc., is transmitted to the electronic medical chart server apparatus. The electronic medical chart server apparatus transmits the received information to the medical information storage apparatus 2200. The aforementioned information includes a patient ID for identifying a subject to be diagnosed, collector information on a collector of information (e.g., an attending doctor, a nurse, etc.), event information on an event from which the information was collected (e.g., a medical examination performed before a chest X-ray CT test), and time information on a time of collection (e.g., Feb. 3, 20XX 9:30 a.m.). The information received by the medical information storage apparatus 2200 is then stored as medical information in the medical database 2210.

The nursing chart system 2320 is an information system that manages nursing charts for recording nursing contents. The nursing chart system 2320 includes, for example, a nursing chart server apparatus and a nursing chart terminal apparatus.

The nursing chart server apparatus is a computer apparatus that performs processing related to a nursing chart. The nursing chart server apparatus is composed of one or more computer apparatuses, depending on the scale of the system. The nursing chart terminal apparatus is used by a nurse, etc., who inputs and refers to the nursing chart. One or more nursing chart terminal apparatuses are provided, depending on the scale of the system. The nursing chart server apparatus and the nursing chart terminal apparatus are connected to the network N.

Information input by a nurse, etc., via the nursing chart terminal apparatus is transmitted to the nursing chart server apparatus. The nursing chart server apparatus transmits the received information to the medical information storage apparatus 2200. The aforementioned information includes the patient ID, the collector information, the event information, free text, numerical items such as test values, Booleans (True/False), the time information, etc. The information received by the medical information storage apparatus 2200 is then stored as medical information in the medical database 2210.

The consultation record system 2330 is an information system that manages consultation records for recording consultation contents. The consultation record system 2330 includes, for example, a consultation record server apparatus and a consultation record terminal apparatus.

The consultation record server apparatus is a computer apparatus that performs processing related to the consultation record. The consultation record server apparatus is composed of one or more computer apparatuses, depending on the scale of the system. The consultation record terminal apparatus is used by a cancer counselor, etc., who inputs and refers to the consultation record. One or more consultation record terminal apparatuses are provided, depending on the scale of the system. The consultation record server apparatus and the consultation record terminal apparatus are connected to the network N. Information input by a cancer counselor, etc., via the consultation record terminal apparatus is transmitted to the consultation record server apparatus. The consultation record server apparatus transmits the received information to the medical information storage apparatus 2200. The aforementioned information includes the patient ID, the collector information, the event information, free text, numerical items such as test values, Booleans (True/False), the time information, etc. The information received by the medical information storage apparatus 2200 is then stored as medical information in the medical database 2210.

The radiology information system (RIS) 34 is an information system that manages information at a radiology department in a hospital, and includes a radiology information server apparatus and a radiology information terminal apparatus.

The radiology information server apparatus is a computer apparatus that performs processing related to management of information in the radiation department. The radiology information server apparatus is composed of one or more computer apparatuses, depending on the scale of the system. The radiology information terminal apparatus is used by, e.g., a radiation technologist at a radiation department. One or more radiology information terminal apparatuses are provided, depending on the scale of the system. The radiology information server apparatus and the radiology information terminal apparatus are connected to the network N.

Information input by a radiological technologist, etc., via the radiology information terminal apparatus is transmitted to the radiology information server apparatus. The radiology department server apparatus transmits the received information to the medical information storage apparatus 2200. The aforementioned information includes the patient ID, the collector information, the event information, free text, numerical items such as test values, Booleans (True/False), the time information, etc. The information received by the medical information storage apparatus 2200 is then stored as medical information in the medical database 2210.

The laboratory information system (LIS) 2350 is an information system that manages information at a clinical laboratory department in a hospital, and includes a laboratory information server apparatus, a laboratory information terminal apparatus, and a test device.

For example, in the laboratory information system 2350, a clinical laboratory technician performs a specimen test or a physiological function test based on order information transmitted from the electronic medical chart system 2310. The specimen test may be entrusted to an external inspection institution such as a health inspection station.

The laboratory information server apparatus is a computer apparatus that performs processing related to management of information in a clinical test. The laboratory information server apparatus is composed of one or more computer apparatuses, depending on the scale of the system. The laboratory information terminal apparatus is used by, e.g., a clinical laboratory technologist at a clinical laboratory department. One or more laboratory information terminal apparatuses are provided, depending on the scale of the system. The laboratory information server apparatus and the laboratory information terminal apparatus are connected to the network N.

The test device is a device related to execution of a specimen test in which a sample such as blood, urine, etc., obtained from a patient is analyzed by a clinical laboratory technician, and a physiological function test in which the patient's electroencephalogram, electrocardiogram, etc., are measured. Examples of the specimen test include a pathology test, a blood/biochemical test, a general test for, e.g., urine or excrement, an immunoserum test, a microbial test, a test related to blood transfusion, organ transplant, etc.

Examples of the physiological function test include an electroencephalogram test, a respiratory function test, a cardiac system test, a fundus photographic test, etc. The plurality of test devices is installed according to the type of specimen test and the type of physiological function test. Meanwhile, at least a part of the specimen test may be performed by an external testing institution. In such a case, the laboratory information server apparatus receives and stores test results related to the specimen test from such an external testing institution. Furthermore, the laboratory information server apparatus transmits the test results to the electronic medical chart system 2310.

Information input by a clinical laboratory technologist, etc., via the laboratory information terminal apparatus is transmitted to the laboratory information server apparatus. The laboratory information server apparatus transmits the received information to the medical information storage apparatus 2200. The aforementioned information includes the patient ID, the collector information, the event information, free text, numerical items such as test values, Booleans (True/False), the time information, etc. The information received by the medical information storage apparatus 2200 is then stored as medical information in the medical database 2210.

The pharmaceutical service support system 2360 is an information system that manages information related to pharmaceutical operations (for example, creation of drug prescription records, etc.). The pharmaceutical service support system 2360 includes, for example, a pharmaceutical service support server apparatus and a pharmaceutical service support terminal apparatus.

The pharmaceutical service support server apparatus is a computer apparatus that performs processing related to pharmaceutical services. The pharmaceutical service support server apparatus is composed of one or more computer apparatuses, depending on the scale of the system. The pharmaceutical service support terminal apparatus is used by a pharmacist, etc., who inputs and refers to drug prescription records, etc. One or more pharmaceutical service support terminal apparatuses are provided, depending on the scale of the system. The pharmaceutical service support server apparatus and the pharmaceutical service support terminal apparatus are connected to the network N.

Information input by a pharmacist, etc., via the pharmacy service support terminal apparatus is transmitted to the pharmaceutical service support server apparatus. The pharmaceutical service support server apparatus transmits the received information to the medical information storage apparatus 2200. The aforementioned information includes the patient ID, the collector information, the event information, free text, numerical items such as test values, Booleans (True/False), the time information, etc. The information received by the medical information storage apparatus 2200 is then stored as medical information in the medical database 2210.

A surgical support system 2370 is an information system that manages information related to surgery. For example, the surgical support system 2370 stores and manages a surgical implementation report, etc., that records information on the implemented surgery. The surgical support system 2370 includes, for example, a surgical support server apparatus and a surgical support terminal apparatus.

The surgical support server apparatus is a computer apparatus that performs processing related to surgery. The surgical support server apparatus is composed of one or more computer apparatuses, depending on the scale of the system. The surgical support terminal apparatus is used by a surgeon, etc., who inputs and refers to a surgical performance report, etc. One or more surgical support terminal apparatuses are provided, depending on the scale of the system. The surgical support server apparatus and the surgical support terminal apparatus are connected to the network N.

Information input by a surgeon, etc., via the surgical support terminal apparatus is transmitted to the surgical support server apparatus. The surgical support server apparatus transmits the received information to the medical information storage apparatus 2200. This information includes a patient ID, collector information, event information, time information, etc. The information received by the medical information storage apparatus 2200 is then stored as medical information in the medical database 2210.

The nutrition management system 2380 is an information system that manages information on a nutritional status of a subject. The nutrition management system 2380 includes, for example, a nutrition management server apparatus and a nutrition management terminal apparatus.

The nutrition management server apparatus is a computer apparatus that performs processing related to nutrition management of a subject. The nutrition management server apparatus is composed of one or more computer apparatuses, depending on the scale of the system. The nutrition management terminal apparatus is used by a nutritionist, etc., who inputs and refers to information indicating, e.g., a subject's nutrition intake record. One or more nutrition management terminal apparatuses are provided, depending on the scale of the system. The nutrition management server apparatus and the nutrition management terminal apparatus are connected to the network N.

Information input by a nutritionist, etc., via the nutrition management terminal apparatus is transmitted to the nutrition management server apparatus. The nutrition management server apparatus transmits the received information to the medical information storage apparatus 2200. The aforementioned information includes the patient ID, the collector information, the event information, free text, numerical items such as test values, Booleans (True/False), the time information, etc. The information received by the medical information storage apparatus 2200 is then stored as medical information in the medical database 2210.

The patient daily life information system 2390 is an information system that manages a patient's daily information related to a patient's daily life. The patient's daily life information may include personal health record (PHR), etc. The patient daily life information system 2390 includes, for example, a patient daily life information server apparatus and a patient daily life information terminal apparatus. The patient daily life information system 2390 may include a wearable device capable of measuring a patient's electrocardiogram, pulse, etc.

The patient daily life information server apparatus is a computer apparatus that performs processing related to patient daily life information. The patient daily life information server apparatus is composed of one or more computer apparatuses, depending on the scale of the system. The patient daily life terminal apparatus is used by a nurse, etc., who inputs and refers to information indicating of, e.g., a patient's health condition. One or more patient daily life information terminal apparatuses are provided, depending on the scale of the system. The patient daily life information server apparatus and the patient daily life information terminal apparatus are connected to the network N.

Information input by a nurse, etc., via the patient daily life terminal apparatus is transmitted to the patient daily life server apparatus. The patient daily life server apparatus transmits the received information to the medical information storage apparatus 2200. This information includes a patient ID, collector information, event information, time information, etc. The information received by the medical information storage apparatus 2200 is then stored as medical information in the medical database 2210.

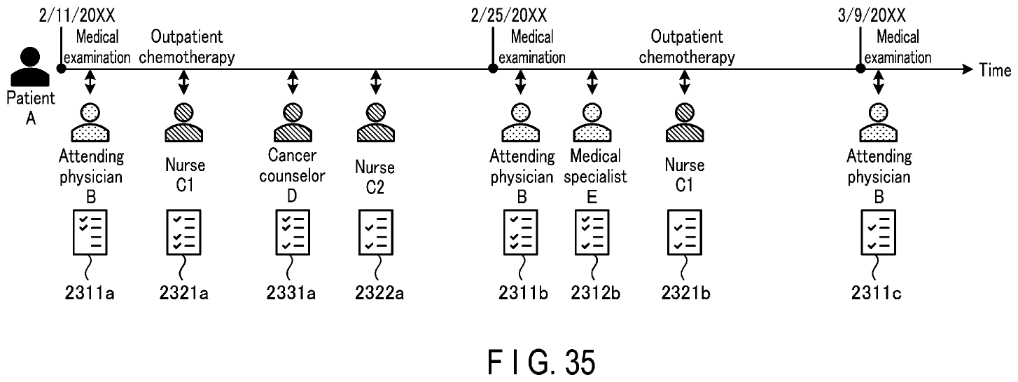

Herein, processing of registering medical information in the medical database 2210 will be described with reference to FIG. 35. FIG. 35 is a diagram for explaining one example of processing of registering medical information in the medical database 2210.

In FIG. 35, a date such as “2/11/20XX” represents a date and time of collection of medical information. “Patient A” is information indicating a patient's name. “Medical examination”, “outpatient chemotherapy”, etc., represent an event in which information was collected. In addition, “attending physician B”, “nurse C1”, “cancer counselor”, etc., represent an information collector.

For example, the attending physician B examines a patient A on 2/11/20XX, and information on the patient A obtained in the medical treatment is input along with a patient ID, collector information, event information, free text, numerical items such as test values, Booleans (True/False), time information, etc., into an electronic medical chart using the electronic medical chart terminal apparatus. In FIG. 35, the patient ID corresponds to a patient ID of patient A. The collector information corresponds to a name of the attending physician B, a staff ID of the attending physician B, etc. Furthermore, the event information corresponds to information indicating a medical examination. The time information is 2/11/20XX.

The input information is then transmitted as medical information 2311a to the medical information storage apparatus 2200 by the electronic medical chart server apparatus. The medical information storage apparatus 2200 registers medical information 2311a in the medical database 2210. Medical information 2321a, 2331a, 2322a, 2311b, 2312a, 2321b, and 2311c is also registered in the medical database 2210 through the same processing as that described above.

Referring back to FIG. 34, the configuration of the diagnosis support apparatus 2100 will be described. The diagnosis support apparatus 2100 is a computer such as a personal computer (PC), a server apparatus, etc. The diagnosis support apparatus 2100 may be provided in a cloud environment, or may be installed in, e.g., a medical institution such as a hospital or a facility of a business operator providing healthcare services.

The diagnosis support apparatus 2100 includes a network (NW) interface 2110, a memory circuit 2120, an input interface 2130, a display 2140, and a processing circuit 2150.

The NW interface 2110 is connected to the processing circuit 2150 and controls the transmission and communications of various types of data between the diagnosis support apparatus 2100, the medical information storage apparatus 2200, and the medical information input support system 2300. The NW interface 2110 is realized by a network card, a network adapter, a network interface controller (NIC), etc.

The memory circuit 2120 is connected to the processing circuit 2150 and stores various items of information and programs used in the processing circuit 2150.

The memory circuit 2120 is realized by, for example a semiconductor memory element such as a random-access memory (RAM) or a flash memory, a hard disk, an optical disk, etc. The memory circuit 2120 is also referred to as a storage.

The input interface 2130 includes a trackball, a switch button, a mouse, a keyboard, a touch pad that allows input operation to be performed by touching the operation surface, a touch screen in which the display screen and the touch pad are integrated, a non-contact input circuit using an optical sensor, a voice input circuit, etc. The input interface 2130 is connected to the processing circuit 2150, and converts an input operation received from a user into an electrical signal and outputs it to the processing circuit 2150.

The display 2140 is a liquid crystal display, an organic electro-luminescence (OEL) display, and the like. The input interface 2130 and the display 2140 may be integrated. For example, the input interface 2130 and the display 2140 may be realized by a touch panel. The display 2140 is an example of the display.

The processing circuit 2150 is a processor that realizes a function corresponding to each program by reading and executing a program from the memory circuit 2120. The processing circuit 2150 according to the present embodiment includes an acquisition function 2151, a calculation function 2152, a specifying function 2153, an analysis function 2154, and a display control function 2155. Herein, the acquisition function 2151 is an example of an acquirer. The calculation function 2152 is an example of a calculator. The specifying function 2153 is an example of a second specifier. The analysis function 2154 is an example of a first specifier and an analyzer. The display control function 2155 is an example of a display controller.

Herein, for example, each of the processing functions of the acquisition function 2151, the calculation function 2152, the analysis function 2154, the specifying function 2153, and the display control function 2155, which are structural elements of the processing circuit 2150, are stored in the form of a program that can be executed by a computer, in the memory circuit 2120.

The processing circuit 2150 is, for example, a processor. For example, the processing circuit 2150 reads out each program from the memory circuit 2120 and executes it to realize the function corresponding to the program. In other words, the processing circuit 2150 in the state of having read out respective programs acquires respective functions shown within the processing circuit 2150 of FIG. 34.

FIG. 34 assumes that the single processor realizes the processing functions performed by the acquisition function 2151, the calculation function 2152, the specifying function 2153, the analysis function 2154, and the display control function 2155; however, the processing circuit 2150 may be configured by a combination of a plurality of independent processors, and the functions may be realized by the processors executing the programs.

Although FIG. 34 illustrates the single memory circuit 2120 storing a program corresponding to respective processing functions, a plurality of memory circuits may be provided in a distributed manner and the processing circuit 2150 may be configured to read a program from a corresponding circuit.

In the above descriptions, an example has been described in which the “processor” reads out a program corresponding to each function from the memory circuit and executes it; however, the embodiment is not limited to this example. The term “processor” used in the present embodiment refers to, for example, a central processing unit (CPU), a graphics processing unit (GPU), or a circuit such as an application specific integrated circuit (ASIC), or a programmable logic device (e.g., a simple programmable logic device (SPLD), a complex programmable logic device (CPLD), or a field programmable gate array (FPGA)).

In the case of the processor being a CPU, for example, the processor realizes a function by reading and performing a program stored in a storage circuit. On the other hand, if the processor is an ASIC, the function corresponding to a program is directly incorporated into a circuit of the processor as a logic circuit, instead of being stored in the memory circuit 2120.

Each processor in the present embodiment is not limited to a single circuit-type processor, and multiple independent circuits may be combined and integrated as a single processor to realize the intended functions. Furthermore, multiple components or features as given in FIG. 34 may be integrated as one processor to realize the respective functions.

The acquisition function 2151 acquires medical information collected as medical data by a plurality of healthcare professionals. For example, the acquisition function 2151 refers to the medical database 2210 via the NW interface 2110 and acquires medical information on a target subject. The acquisition function 2151 may acquire medical information from the electronic medical chart server apparatus of the electronic medical chart system 2310, etc., via the NW interface 2110.

The calculation function 2152 calculates, based on the medical data, a feature amount in the medical data for a predetermined period. The present embodiment uses, as a feature amount, an accumulated value or a fluctuation value indicating the magnitude of fluctuation between pieces of medical information of the same type. The calculation function 2152 according to the present embodiment calculates a fluctuation value indicating the magnitude of fluctuation between pieces of medical information of the same type for a plurality of pieces of medical information related to the same subject acquired by the acquisition function 2151.

For example, the calculation function 2152 calculates a first fluctuation value indicating the magnitude of fluctuation in medical information acquired by the acquisition function 2151, between collectors of the information. For example, the calculation function 2152 calculates a second fluctuation value indicating the magnitude of temporal fluctuation in information. For example, the calculation function 2152 calculates a third fluctuation value indicating the magnitude of fluctuation between medical events in which information was collected. For example, the calculation function 2152 calculates an overall fluctuation value from the first fluctuation value to the third fluctuation value.

Herein, processing of calculating a fluctuation value calculation performed by the calculation function 2152 will be described. First, processing of calculating the first fluctuation value will be described. The following will describe an example case in which the attending physician B, a nurse C1, a nurse C2, and a cancer counselor D each collect information relating to a patient A's thought about knowing his or her life expectancy.

First, regarding the knowledge of the life expectancy, the information is subjective information on a subject, and the magnitude of fluctuation cannot be simply evaluated. Therefore, for subjective information on the subject, such as information related to the subject's emotions, a method of evaluating the subjective information is defined.

Hereinafter, a method of evaluating knowledge of a patient's life expectancy will be described. For example, based on the contents of interviews with a patient, each information collector evaluates the degree of the patient's desire to know his or her life expectancy on five levels, “0: no desire to know, 1: prefers not to know, 2: no idea, 3: prefers to know, and 4: wants to know.” Each information collector then inputs an evaluation result of the patient's desire to know his or her patient's life expectancy using a terminal apparatus, etc., of the medical information input support system 2300. In this manner, information relating to the knowledge of the patient's life expectancy can be quantified. Herein, the quantified information relating to the knowledge of the patient's life expectancy is an example of an index related to medical information.

In this example, information relating to the knowledge of the patient's life expectancy is quantified through input by each information collector; however, information relating to the knowledge of the patient's life expectancy may be quantified by extracting and evaluating it using existing natural language processing technology.

Based on the above premise, for example, assume that on the date o/o, with respect to the patient A's desire to know his or her life expectancy, the attending physician B makes the evaluation of “4: rather wants to know”, and similarly, the nurse C1 also makes the evaluation of “4: rather wants to know”. Similarly, assume that the nurse C2 makes the evaluation of “0: no desire to know” and the cancer counselor D makes the evaluation of “1: prefers not to know”.

In this case, for example, the calculation function 2152 calculates a standard deviation in the evaluations by the attending physician B, the nurse C1, the nurse C2, and the cancer counselor D. The calculation function 2152 sets the calculated standard deviation divided by 2.1 to a first fluctuation value.

The first fluctuation value may be not limited to the standard deviation but may also be another statistic. For example, the calculation function 2152 may use a value of variance in the evaluations by the attending physician B, the nurse C1, the nurse C2, and the cancer counselor D, as the first fluctuation value. Furthermore, for example, the calculation function 2152 calculates a difference between the maximum value and the average value and a difference between the minimum value and the average value of the evaluations by the attending physician B, the nurse C1, the nurse C2, and the cancer counselor D, and may set a value of the larger difference to the first fluctuation value.

As another example, the following will describe a case in which the attending physician B, the nurse C1, the nurse C2, the and cancer counselor D each collect information on the physical pain of patient A.

First, a method of evaluating the patient's physical pain will be described. For example, based on the contents of interviews with a patient, each information collector evaluates the patient's pain on five levels, “0: none, 1: light, 2: slightly painful, 3: painful, 4: extremely painful”. Each information collector then inputs an evaluation result of the patient's physical pain using a terminal apparatus, etc., of the medical information input support system 2300. In this manner, information on the patient's physical pain can be quantified. Herein, the quantified information on the patient's physical pain is an example of an index related to medical information.

In this example, information on the patient's physical pain is quantified through input by each information collector; however, information on the patient's physical pain may be quantified by extracting and evaluating it using existing natural language processing technology.

Based on the above premise, for example, assume that on the date o/o, with respect to the patient A's physical pain, the attending physician B makes the evaluation of “4: extremely painful”, and similarly, the nurse C1 also makes the evaluation of “4: extremely painful”. Similarly, assume that the nurse C2 makes the evaluation of “0: none”, and the cancer counselor D makes the evaluation of “1: light”.

In this case, for example, the calculation function 2152 calculates a standard deviation in the evaluations by the attending physician B, the nurse C1, the nurse C2, and the cancer counselor D. The calculation function 2152 sets the calculated standard deviation divided by 2.1 to a first fluctuation value.

Next, processing of calculating the second fluctuation value will be described. As an example, the following will describe a case in which the attending physician B collects information on the patient A's physical pain on different dates, ∘/∘, ∘/Δ, and ∘/□.

For example, assume that the attending physician B evaluates the physical pain as “0: none” on the date ∘/∘, as “1: light” on the date ∘/Δ, and as “4: extremely painful” on the date ∘/□.

In this case, for example, the calculation function 2152 calculates the difference between the minimum value and the maximum value of the evaluations made on the dates ∘/∘, ∘/Δ, and ∘/□. Then, the calculation function 2152 calculates, as the second fluctuation value, the difference between the minimum value and the maximum value of the evaluations made on the dates ∘/∘, ∘/Δ, and ∘/□, which is calculated as 4−0=4.

The second fluctuation value may be not limited to the difference between the minimum value and the maximum value of the evaluations but may also be another statistic, etc. For example, the calculation function 2152 may use a value of distribution among the dates ∘/∘, ∘/Δ, and ∘/□ as the second fluctuation value.

Furthermore, for example, the calculation function 2152 may calculate the difference between the evaluation made on the date ∘/∘ and the evaluation made on the date ∘/Δ, and the difference between the evaluation made on the date ∘/Δ and the evaluation made on the date ∘/□, and sets a value of the larger difference to the second fluctuation value. Furthermore, for example, the calculation function 2152 may calculate a value of distribution between the difference between the evaluation made on the date ∘/∘ and the evaluation made on the date ∘/Δ, and the difference between the evaluation made on the date ∘/Δ and the evaluation made on the date ∘/□, and may set this value of distribution to the second fluctuation value.

Next, processing of calculating a third fluctuation value will be described. As an example, the following will describe a case in which each information collector collects information on physical pain through a medical examination, outpatient chemotherapy, chest CT scan, and counseling.

For example, assume that the attending physician B evaluated the physical pain as “4: extremely painful” through a medical examination conducted on the date ∘/∘, and the nurse C1 evaluated the physical pain as “4: extremely painful” through the outpatient chemotherapy conducted on the date ∘/Δ. Furthermore, assume that through the chest CT scan conducted on the date ∘/□, the nurse C2 evaluated the physical pain as “1: light”. In addition, assume that through the counseling conducted on the date ∘/x, the cancer counselor D evaluated the physical pain as “0: none”.

In such a case, for example, the calculation function 2152 calculates the standard deviation of evaluations from the medical examination, the outpatient chemotherapy, the chest CT examination, and the counseling. The calculation function 2152 then sets the calculated standard deviation divided by 2.06 to the third fluctuation value.

The third fluctuation value may be not limited to the standard deviation but may be another statistic, etc. For example, the calculation function 2152 may use, as the third fluctuation value, a value of deviation of evaluations from the medical examination, the outpatient chemotherapy, the chest CT examination, and the counseling.

Furthermore, for example, the calculation function 2152 may calculate a difference between the maximum value and the average value and a difference between the minimum value and the average value of the evaluations from the medical examination, the outpatient chemotherapy, the chest CT examination, and the counseling, and may set a value of the larger difference to the third fluctuation value.

Next, processing of calculating an overall fluctuation value will be described. For example, the calculation function 2152 sums the first fluctuation value, the second fluctuation value, and the third fluctuation value, and calculates the overall fluctuation value. Meanwhile, the calculation function 2152 may use an average value of the first fluctuation value, the second fluctuation value, and the third fluctuation value, as the overall fluctuation value.

The calculation function 2152 may calculate the first fluctuation value, the second fluctuation value, and the third fluctuation value using the degree of fluctuation in information on a patient other than a target patient. For example, the calculation function 2152 may normalize a statistic, etc., indicating the degree of fluctuation calculated for a target patient individual with a statistic, etc., indicating the degree of fluctuation calculated for a total of multiple patients, and may use a value obtained by this normalization as each of the first fluctuation value, the second fluctuation value, and the third fluctuation value. In this manner, the relative degree of fluctuation of the target patient can be expressed as each of the first fluctuation value, the second fluctuation value, and the third fluctuation value.

The specifying function 2153 specifies the type (category) of medical information for each item of medical information acquired by the acquisition function 2151. In the present embodiment, the specifying function 2153 specifies which one of the categories of “medical indication”, “patient's view and preference”, “QOL”, and “contextual features” the medical information acquired by the acquisition function 2151 belongs to.

“Medical indication” is, for example, medical information on, e.g., the size of a cancer and the presence or absence of metastasis. “Patient's view and preference” is, for example, information on the subject's intentions, such as whether the subject emphasizes breast preservation or the treatment effect in the case of breast cancer. “QOL” is information on the subject's daily life and quality of life. “Contextual features” is information on the surroundings of the subject, such as family history.

For example, the specifying function 2153 refers to a category-specifying table and specifies which one of the categories of “medical indication”, “patient's view and preference”, “QOL”, and “contextual features” the medical information belongs to. Hereinafter, the category-specifying table will be described.

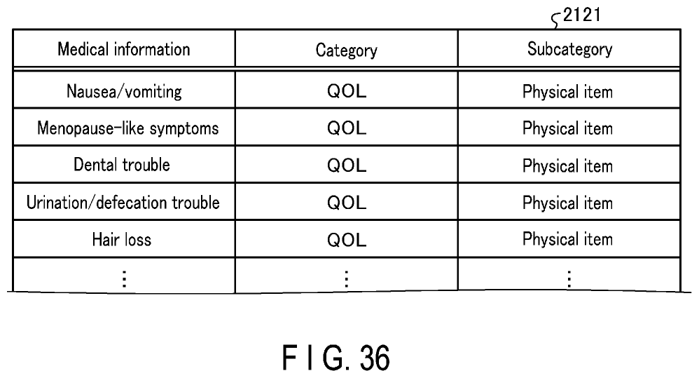

For example, the category-specifying table is a data table that associates medical information with a category. The category-specifying table is stored in, for example, the memory circuit 2120. FIG. 36 is a diagram illustrating an example of a category-specifying table 2121. The category-specifying table 2121 in FIG. 36 is a data table that associates medical information with categories and subcategories.

The medical information represents information obtained from a patient. The category represents a category of medical information. The subcategory represents a more detailed category than the category of medical information. The subcategory may be the same character string as that of the medical information. The category and subcategory may be referred to as a diagnosis item or a display item. Furthermore, the category may be referred to as a major item.

For example, the first row of the category specifying table 2121 in FIG. 36 indicates that medical information related to the subject's “nausea/vomiting” belongs to the category “QOL” and is further classified into the subcategory of “physical item”.

Referring back to FIG. 34, the description will continue. The analysis function 2154 performs processing of analyzing the medical information acquired by the acquisition function 2151.

For example, the analysis function 2154 analyzes the degree of fluctuation magnitude for each category of medical information specified by the specifying function 2153. For example, the analysis function 2154 calculates an average value of the overall fluctuation values calculated by the calculation function 2152 for each category of medical information. The analysis function 2154 classifies the degree of fluctuation magnitude into three levels for each category of medical information based on the average value.

Furthermore, for example, the analysis function 2154 analyzes the degree of fluctuation magnitude for each item of medical information based on the magnitude of the fluctuation value calculated by the calculation function 2152. For example, the analysis function 2154 classifies the degree of fluctuation magnitude into five levels for each item of medical information according to the magnitude of the overall fluctuation value calculated by the calculation function 2152.

Furthermore, for example, the analysis function 2154 analyzes the fluctuation value calculated by the calculation function 2152 and specifies the type of fluctuation. For example, in the case where the first fluctuation value calculated by the calculation function 2152 exceeds a predetermined threshold, the analysis function 2154 specifies the type of fluctuation as a systematic fluctuation between information collectors.

For example, in the case where the second fluctuation value calculated by the calculation function 2152 exceeds a predetermined threshold value, the analysis function 2154 specifies the type of fluctuation as a temporal fluctuation. For example, in the case where the third fluctuation value calculated by the calculation function 2152 exceeds a predetermined threshold value, the analysis function 2154 specifies the type of fluctuation as a systematic fluctuation between events.

For example, in the case where the first fluctuation value and the second fluctuation value exceed the threshold values, the analysis function 2154 specifies the type of fluctuation as a systematic and temporal fluctuation between collectors. For example, in the case where the first fluctuation value and the third fluctuation value exceed the threshold values, the analysis function 2154 specifies the type of fluctuation as a systematic fluctuation between collectors and events.

For example, in the case where the second fluctuation value and the third fluctuation value exceed the threshold values, the analysis function 2154 specifies the type of fluctuation as a temporal and systematic fluctuation between events. For example, in the case where the first fluctuation value, the second fluctuation value, and the third fluctuation value exceed the threshold values, the analysis function 2154 specifies the type of fluctuation as a temporal and systematic fluctuation between information collectors and between events.

For example, in the case where none of the first fluctuation value, the second fluctuation value, and the third fluctuation value exceed the threshold values, the analysis function 2154 specifies the type of fluctuation as a random fluctuation.

Furthermore, for example, the analysis function 2154 specifies a correlation between pieces of the medical information acquired by the acquisition function 2151.

For example, with respect to a plurality of pieces of medical information acquired by the acquisition function 2151, the analysis function 2154 calculates a correlation coefficient of an index indicative of each item of medical information for each pair of pieces of medical information. As the correlation coefficient, for example, Pearson's correlation coefficient, Spearman's rank correlation coefficient, Kendall's rank correlation coefficient, etc., can be used. In the case where the correlation coefficient exceeds a predetermined threshold, the analysis function 2154 specifies that there is a correlation between the pair of pieces of medical information.

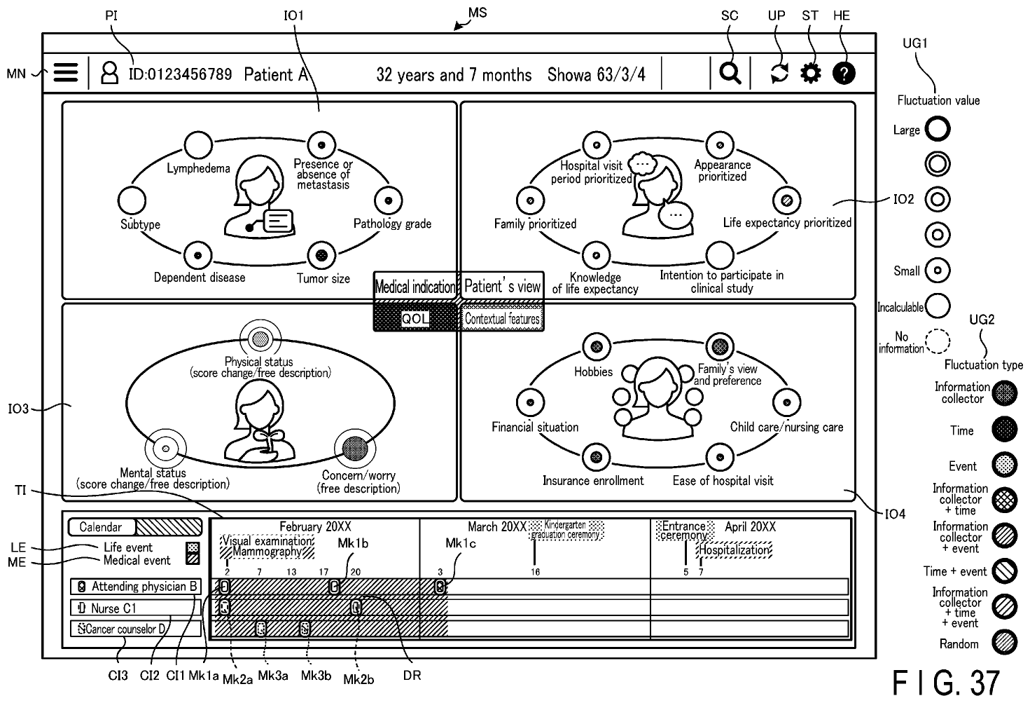

The display control function 2155 causes the displayer to display medical data in a manner according to the feature amount. In the present embodiment, the display control function 2155 causes the display 2140 to display medical information acquired by the acquisition function 2151, along with information indicative of a fluctuation value calculated by the calculation function 2152. Hereinafter, the display control processing of medical information by the display control function 2155 will be described with reference to FIG. 37 to FIG. 47.

FIG. 37 is a diagram showing an example of displaying of medical information according to the present embodiment. FIG. 37 shows a medical information display screen MS for causing the display 2140 to display thereon a summary of medical information.

The medical information display screen MS is comprised of a menu MN, a patient information display area PI, a search button SC, an update button UP, a setting button ST, a help button HE, a medical indication display area IO1, a patient's intention display area IO2, a QOL display area IO3, a contextual feature display area IO4, a life event display area LE, a medical event display area ME, a temporal change display area TI, a data range DR, information collector display areas CI1 to CI3, and marks MK1 to MK3. Furthermore, a legend UG may be displayed on the medical information display screen MS.

The menu MN is a button for, e.g., changing display contents of the medical information display screen MS. For example, in response to a user clicking the menu MN, a button is displayed for switching to a mode in which a correlation between pieces of medical information is displayed. The patient information display area PI is an area for displaying information on a subject. In FIG. 37, a patient ID, a name, an age, and a date of birth of the patient A are displayed.

The search button SC is a button for a user to search for a subject. For example, in response to the user clicking the search button SC, an input form is displayed for inputting search conditions (for example, age, gender, name, etc.) for searching for a subject whose medical information is desired to be displayed.

The update button UP is a button for updating medical information currently displayed to the latest state. The setting button ST is a button for performing display setting (for example, setting of a font used, a font size, a font color, etc.) of the medical information display screen MS. The help button HE is a button for displaying information for assisting a user, such as a Q&A related to the medical information display screen MS.

The medical indication display area IO1 is an area for displaying medical information belonging to medical indications. The example shown in FIG. 37 displays “tumor size”, “pathology grade”, “presence or absence of metastasis”, “lymphedema”, “subtype”, and “dependent disease”, which are subcategories of medical information. The patient's intention display area IO2 is an area for displaying medical information belonging to a patient's view and preference. The example shown in FIG. 37 displays “intention to participate in a clinical study”, “life expectancy prioritized”, “appearance prioritized”, “hospital visit period prioritized”, “family prioritized”, and “knowing life expectancy”.

The QOL display area IO3 is an area for displaying medical information belonging to QOL. The example shown in FIG. 37 displays “physical status (score change/free description)”, “mental status (score change/free description)”, and “concern/worry (free description)”. The contextual feature display area IO4 is an area for displaying medical information belonging to contextual features. The example shown in FIG. 37 displays “ease of hospital visits”, “child care/nursing care”, “family's view and preference”, “hobbies”, “financial situation”, and “insurance enrollment”.

In displaying a summary, only subcategories of medical information are displayed in each of the display areas IO1 to IO4. The display items (subcategories) of each of the above categories are not limited to those. Some of the above display items may not be included, or other display items may be included.

Furthermore, the display control function 2155 changes a display form of a label indicative of a category of medical information according to the magnitude of a fluctuation value of the category of medical information. In the example shown in FIG. 37, a fluctuation in “QOL” is large, a fluctuation in “contextual features” is medium, and fluctuations in “medical indication” and “patient's view and preference” are small, and the degree of these fluctuations is expressed by changing the background color of each label. The above expresses the degree of magnitude of a fluctuation value in each category of medical information in three levels; however, this is not a limitation. For example, the degree of magnitude of the fluctuation value may be expressed in two levels or four levels.

The life event display area LE is an area for displaying information related to a life event of a subject. Life events are events related to the subject's life (for example, his or her child's entrance ceremony, graduation ceremony, etc.). The medical event display area LE is an area for displaying information on a medical event of the subject. A medical event is an event related to a medical treatment of the subject (for example, hospitalization, discharge, examination, surgery, etc.).

The temporal change display area TI is an area for displaying information indicative of a temporal change in medical information. The data range DR indicates a data range of medical information whose fluctuation value is to be displayed.

The example shown in FIG. 37 sets, to the data range DR, the medical information collected by the attending physician B, the nurse C1, and the cancer counselor D during the period from February 2 to Mar. 3, 20XX. This indicates that a user is paying attention to all of the medical information collected by the attending physician B, the nurse C1, and the cancer counselor D during the period from February 2 to Mar. 3, 20XX.

The information collector display areas CI1 to CI3 indicate information collectors who have collected medical information. Each of the marks MK1 to MK3 indicate that a corresponding information collector acquired medical information on the date displayed in the temporal change display area TI. For example, FIG. 37 shows that the attending physician B collected medical information on February 2, February 17, and March 3.

In addition, the “fluctuation value” in the legend UG1 indicates how to express the magnitude of a fluctuation value of each item of medical information. In the example shown in FIG. 37, the fluctuation value increases as the inner circle increases. This makes it clear that, for example, in FIG. 37, the overall view of health of QOL fluctuates greatly. Meanwhile, it is assumed that the fluctuation value of the subcategories of each category of medical information displayed in the summary display shown in FIG. 37 is the maximum value of all of the subcategories; however, the fluctuation value may be an average value or a median value.

Furthermore, the example shown in FIG. 37 shows only one value in the case of no inner circle, which indicates that a fluctuation value cannot be calculated. In the example shown FIG. 37, the outer circle represented by a dotted line indicates that medical information does not exist. By displaying the fact that there is no medical information, a user can easily grasp what kind of information is missing in order to determine a treatment plan, etc.

“Type of fluctuation” in a legend UG2 indicates how to express the type of fluctuation in each item of medical information. In the example of FIG. 37, “type of fluctuation” is represented by a display pattern of an inner circle.

For example, “information collector” represents a display pattern on the medical information display screen MS in the case where a systematic fluctuation is observed between information collections. Similarly, “time” represents a display pattern on the medical information display screen MS in the case where a temporal fluctuation is observed. Similarly, “event” represents a display pattern on the medical information display screen MS in the case where a systematic fluctuation between medical events is observed.

Similarly, “information collector+time” represents a display pattern on the medical information display screen MS in the case where a systematic and temporal fluctuation between information collectors is observed. Similarly, “information collector+event” represents a display pattern on the medical information display screen MS in the case where a systematic fluctuation between information collectors and medical events is observed.

Similarly, “time+event” represents a display pattern on the medical information display screen MS in the case where a temporal fluctuation and a systematic fluctuation between medical events are observed. Similarly, “information collector+time+event” represents a display pattern on the medical information display screen MS in the case where a systematic fluctuation between information collectors, a temporal fluctuation, and a systematic fluctuation between medical events are observed. Similarly, “random” represents a display pattern on the medical information display screen MS in the case where a random fluctuation is observed.

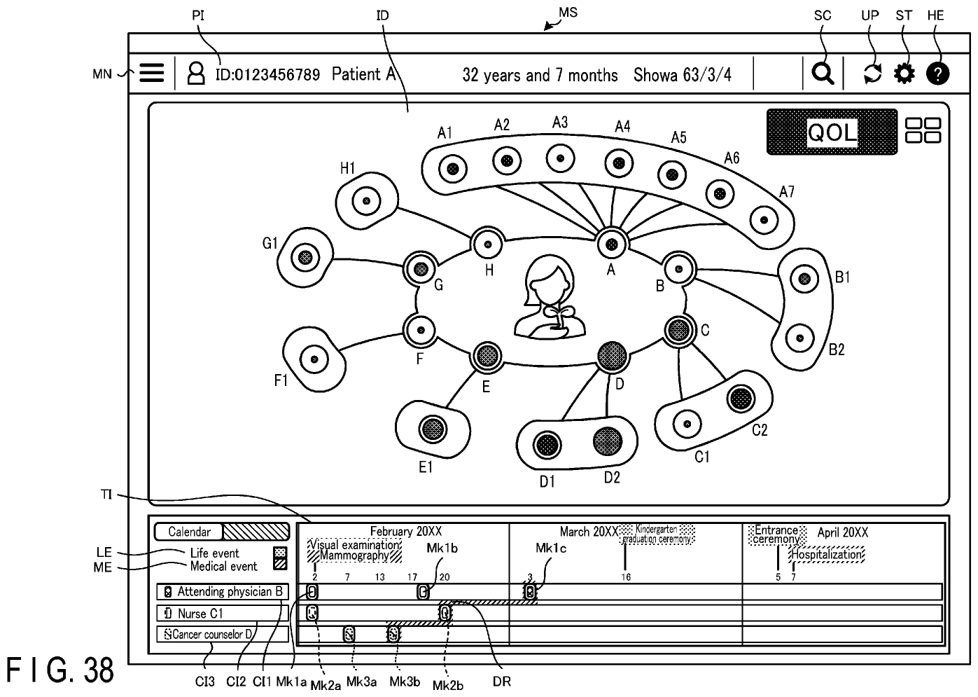

Herein, FIG. 38 is a diagram illustrating an example of displaying of the medical information according to the present embodiment. FIG. 38 shows the medical information display screen MS for causing the display 2140 to display thereon details of the medical information. In displaying details, only medical information belonging to a category of medical information selected by a user is displayed. Furthermore, in displaying details, medical information in the subcategories is also displayed. The medical information display screen MS in FIG. 38 is displayed in the case where, for example, the user clicks the category label with a mouse.

In displaying details, a detailed display area ID of one category is displayed. In FIG. 38, details of the category “QOL” are displayed in the detail display area ID.

The example shown in FIG. 38 sets, to the data range DR, the medical information collected by attending physician B on Mar. 3, 20XX, the medical information collected by the nurse C1 on February 20, and the medical information collected by the cancer counselor D on February 13. This indicates that a user is focusing on a fluctuation between medical information collected by the attending physician B on March 3 and medical information collected by another information collector on the most recent date while centering on the former medical information.

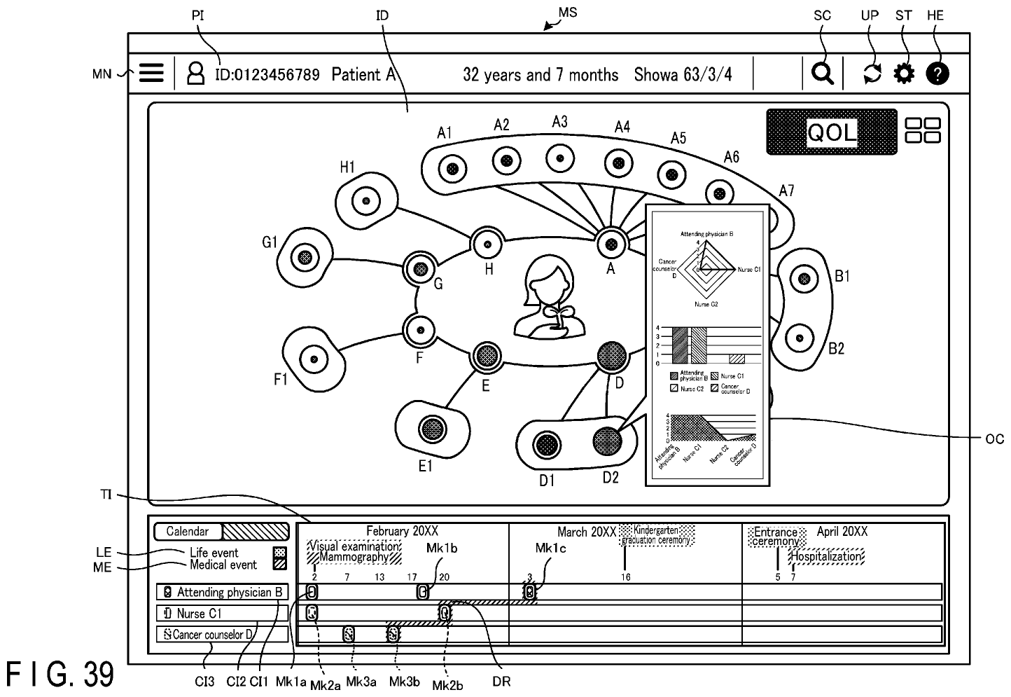

Herein, FIG. 39 is a diagram illustrating an example of displaying of the medical information according to the present embodiment. FIG. 39 is an example of the medical information display screen MS caused to display thereon a detail OC of a fluctuation between information collectors. For example, the detail OC of the fluctuation between information collectors in FIG. 39 is displayed by a user's operation (for example, while the medical information display screen MS in FIG. 38 is displayed, the user selects “displaying detail of fluctuation between information collectors” from a menu displayed by right-clicking “sleep abnormality”, which is one piece of the medical information, with a mouse).

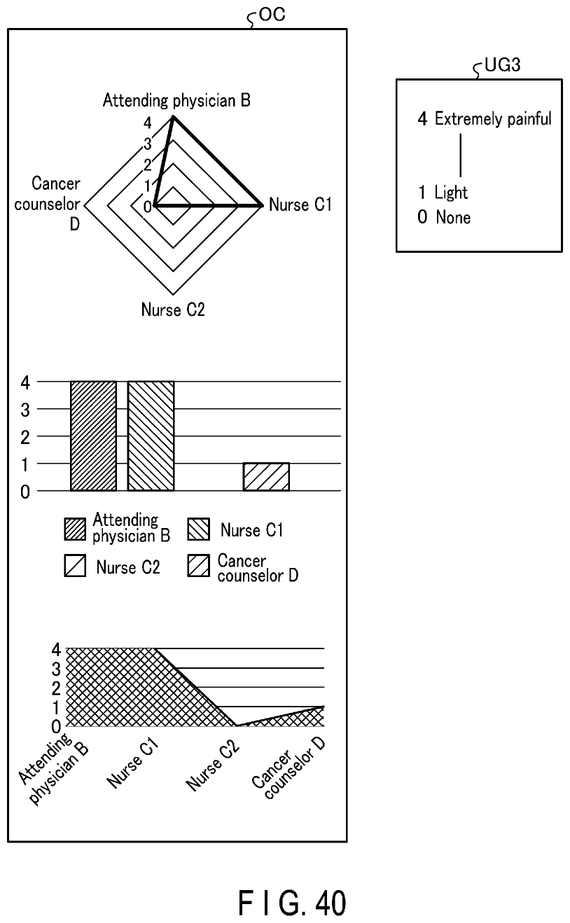

Herein, FIG. 40 is a diagram illustrating an example of displaying of the medical information according to the present embodiment. FIG. 40 is an explanatory diagram obtained by enlarging the detail OC of a fluctuation between the information collectors. A legend UG3 shown in FIG. 40 is information indicating how to view information of the detail OC of a fluctuation between the information collectors. FIG. 40 defines “0” as “none”, “1” as “hard”, and the maximum value as “4”, meaning “extremely painful”, and indicates that a subject feels more pain as the numerical value increases. The legend UG3 may be displayed on the medical information display screen MS along with the detail OC of the fluctuation between the information collectors.

The detail OC of the fluctuation between the information collectors in FIG. 40 compares “sleep abnormality” classified in the subcategory of “overall view of health” between pieces of data collected by the attending physician B, the nurse C1, the nurse C2, and the cancer counselor D. FIG. 40 compares pieces of data collected by the attending physician B, the nurse C1, the nurse C2, and the cancer counselor D in a radar chart, a bar graph, and an area graph.

In this manner, by displaying the details of a fluctuation between the information collectors on the medical information display screen MS, a user can easily check what kind of fluctuation is occurring between the information collectors as well as the magnitude of the fluctuation.

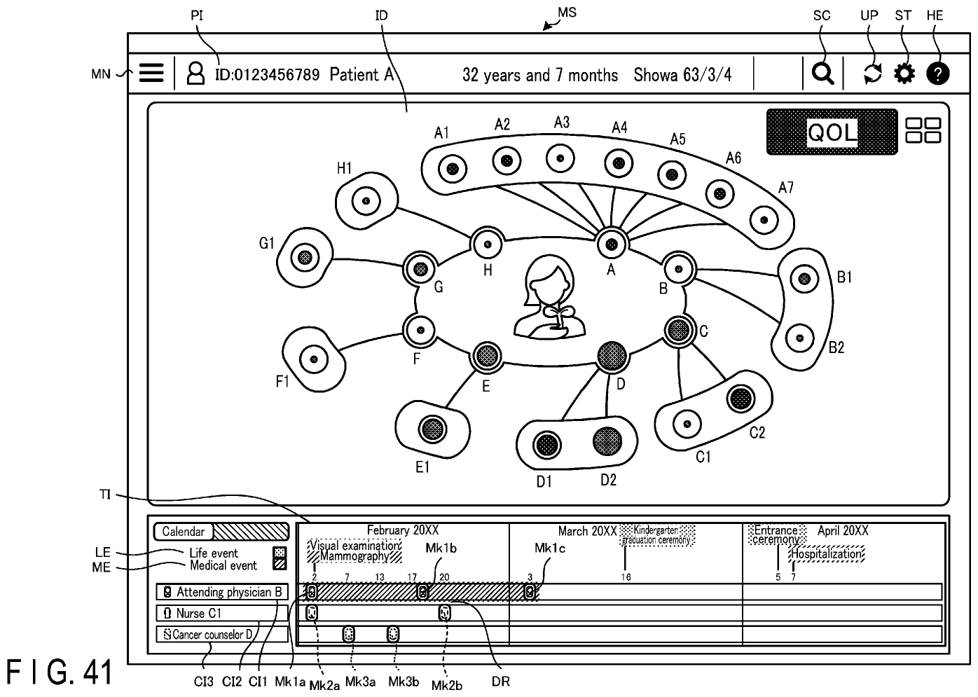

FIG. 41 is a diagram showing an example of displaying of medical information according to the present embodiment. FIG. 41 is the medical information display screen MS that displays thereon the detail display area in which the medical information collected by the attending physician B during the period from February 2 to Mar. 3, 20XX is set to the data range DR. This indicates that a user is paying attention to a temporal change in the medical information collected by the attending physician B during the period from February 2 to Mar. 3, 20XX.

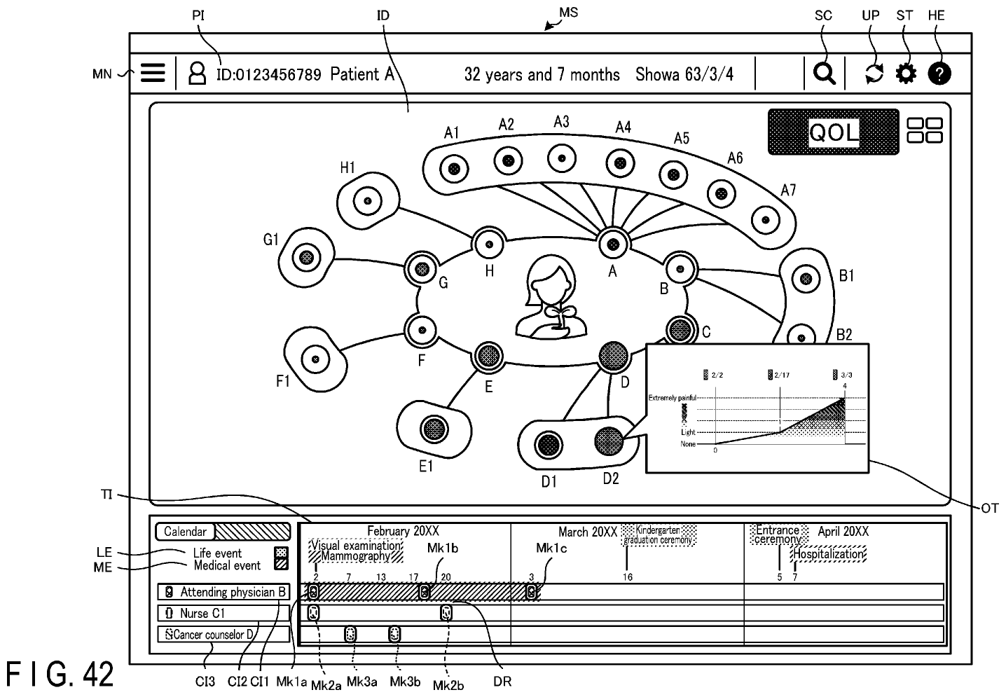

FIG. 42 is a diagram showing an example of displaying of medical information according to the present embodiment. FIG. 42 is an example of the medical information display screen MS in which a detail OT of a temporal fluctuation is displayed. The detail OT of the temporal fluctuation between information collectors in FIG. 42 is displayed by a user's operation (for while the medical information display screen MS in FIG. 41 is displayed, the user selects “displaying detail of temporal fluctuation between information collectors” from a menu displayed by right-clicking “sleep abnormality”, which is one piece of the medical information, with a mouse.

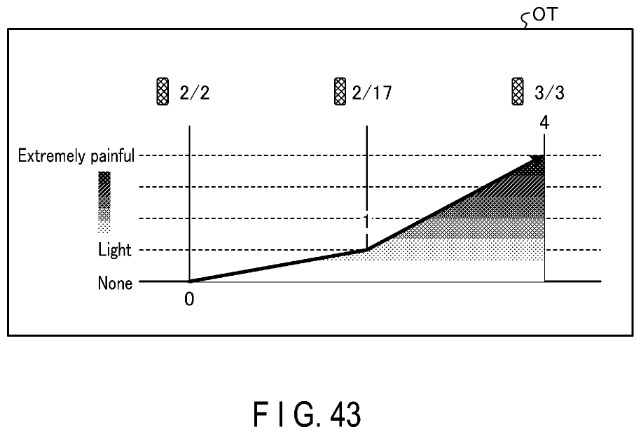

Herein, FIG. 43 is a diagram illustrating an example of displaying of the medical information according to the present embodiment. FIG. 43 is an explanatory diagram obtained by enlarging the detail OT of a temporal fluctuation between the information collectors. The detail OT of the temporal fluctuation in FIG. 43 corresponds to a line graph showing a variation in the evaluation made by the attending physician B from February 2 to Mar. 3, 20XX, with respect to “sleep abnormality” classified in the subcategory of “overall view of health”.

In this manner, by causing the medical information display screen MS to display thereon the displaying of the details of the temporal fluctuation, a user can easily check the temporal change of medical information along with the magnitude of the fluctuation.

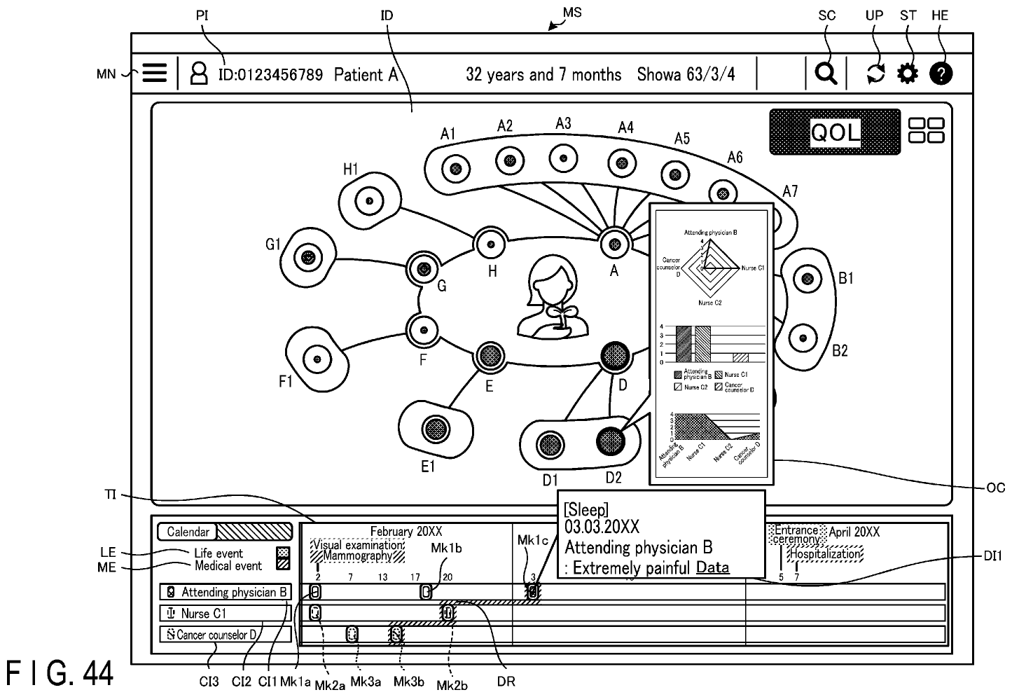

Furthermore, FIG. 44 to FIG. 46 each are a diagram illustrating an example of displaying medical information according to the present embodiment. FIG. 44 to FIG. 46 each are diagrams illustrating an example of displaying details of medical information. FIG. 44 shows the medical information display screen MS in which a detailed display DI′ representing the evaluation of attending physician B on Mar. 3, 20XX is displayed for “sleep abnormality” classified in the subcategory of “overall view of health”.

In the detailed display DI1, “Mar. 3, 20XX” is displayed as the date and time when the medical information was collected, “attending physician B” as the information collector, and “extremely painful” as the content of the medical information. In addition, “Data” represents a link to the electronic description from which the medical information is acquired. This enables a user to check, by clicking “Data”, the contents of the electronic medical chart that is the source of medical information.

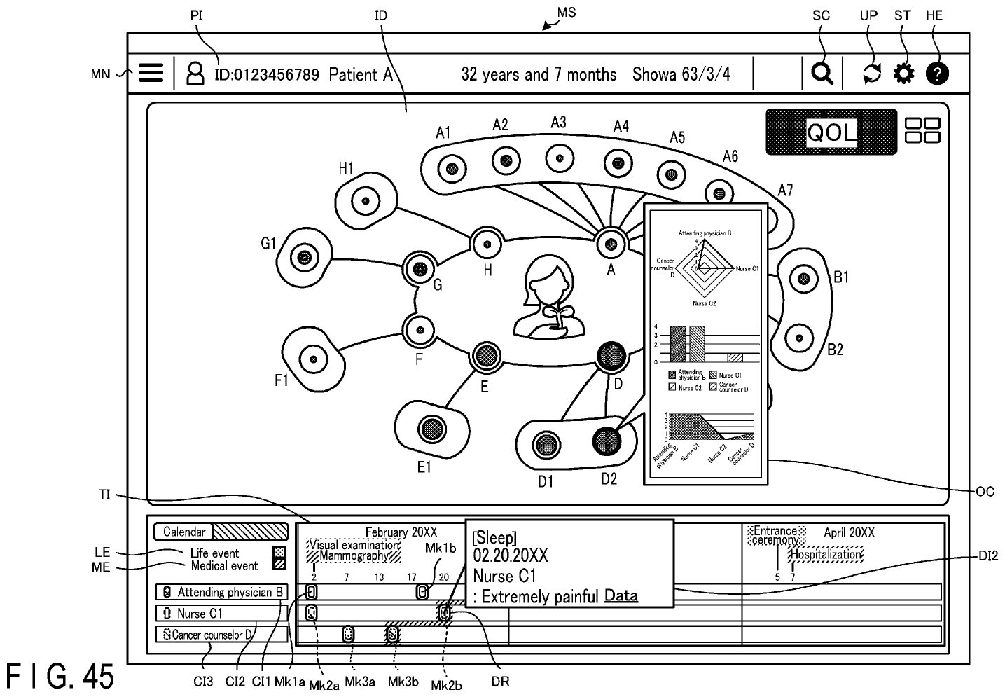

FIG. 45 shows the medical information display screen MS caused to display the detailed display DI2 representing the evaluation made by the nurse C1 on Feb. 20, 20XX for “sleep abnormality” classified in the subcategory of “overall view of health”. In the detailed display DI2, “Feb. 20, 20XX” is displayed as the date and time when the medical information was collected, “nurse C1” is displayed as the information collector, and “terribly painful” is displayed as the content of the medical information. In addition, “Data” represents a link to the nursing chart from which medical information is acquired.

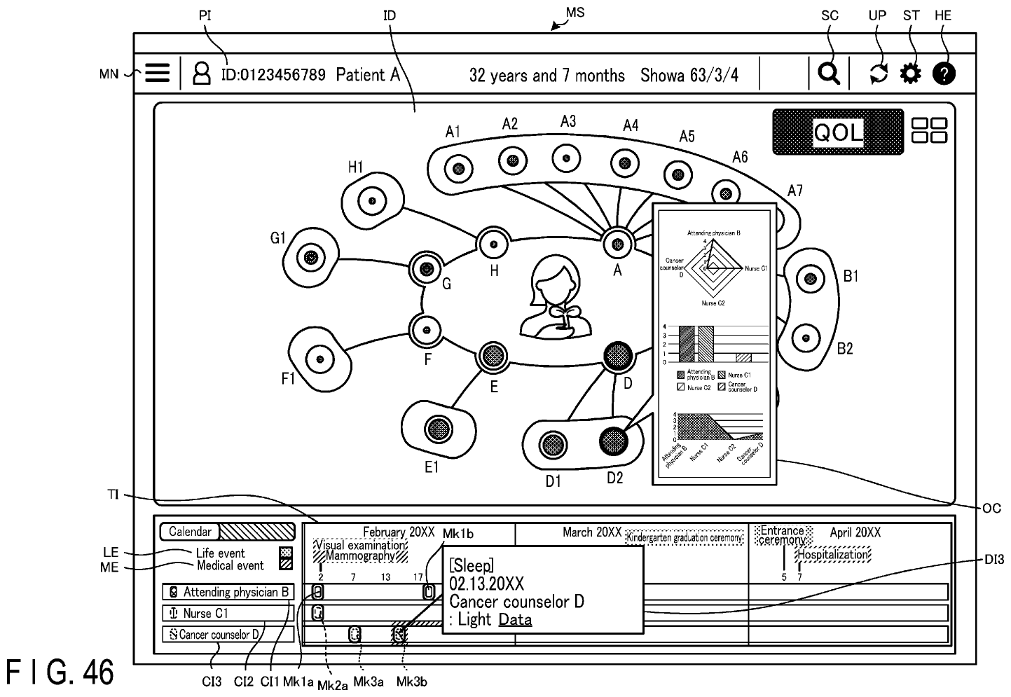

FIG. 46 shows the medical information display screen MS displaying thereon a detailed display DI3 representing the evaluation made by the cancer counselor D on Feb. 13, 20XX for “sleep abnormality” classified in the subcategory of “overall view of health”. In the detailed display DI3, “Feb. 13, 20XX” is displayed as the date and time when the medical information was collected, “cancer counselor D” is displayed as the information collector, and “light” is displayed as the content of the medical information. In addition, “Data” represents a link to the consultation record from which medical information is acquired.

In this manner, by causing the medical information display screen MS to display thereon details, a user can easily check the contents of medical information along with information representing the fluctuation.

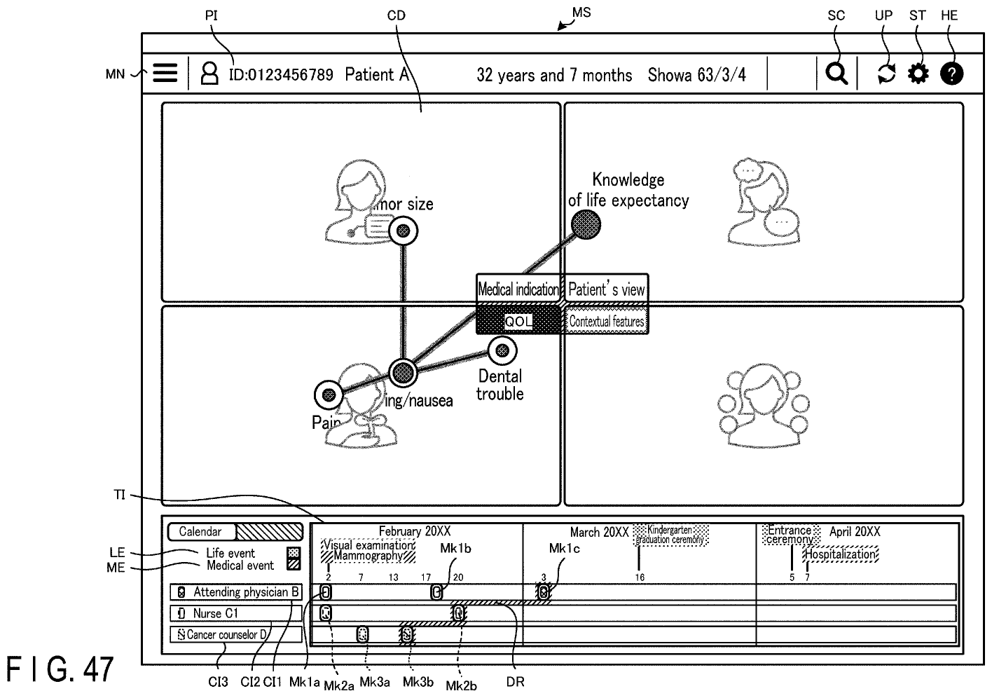

Herein, FIG. 47 is a diagram illustrating an example of displaying the medical information according to the present embodiment. FIG. 47 is an example of a correlation display CD showing a correlation between pieces of medical information. For example, the correlation display CD is displayed in the case where a user selects “display of correlation” from the menu MN.

FIG. 47 focuses on a fluctuation value in “nausea/vomiting” in the subcategory “physical item” in the category of “QOL”, and shows medical information that is correlated with “nausea/vomiting”. It is understood from FIG. 47 that “knowing life expectancy”, “tumor size”, “dental trouble”, and “pain” are correlated with “nausea/vomiting”.

As described above, by displaying the correlation display CD, a user can easily grasp what items are related to each other.

As described above, the processing of displaying the medical information has been described using FIG. 37 to FIG. 47; however, the medical information to be displayed is not limited to the information listed in FIG. 37 to FIG. 47. For example, in a questionnaire on ease of living ([searched on Aug. 4, 2022], Internet <http://gankanwa.umin.jp/pdf/hamamatsulife.pdf>), items stipulated in FACT-B or EORTC QLQ-BR23, etc., which are QOL scales for cancer patients, may also be used as medical information belonging to “QOL” to be displayed. In such a case, the calculation function 2152 may calculate a fluctuation value using the evaluation index of each item specified in the questionnaire on ease of living, FACT-B, EORTC QLQ-BR23, etc.

Furthermore, information defined by a user may be used as the medical information to be displayed. In addition, in the case where a user defines subjective information of a subject (for example, information indicating the subject's emotions such as joy, anger, sadness, etc.) as the medical information to be displayed, he or she defines an evaluation index of the subjective information, too. This enables the calculation function 2152 to calculate the fluctuation value. Therefore, a user can use, as the medical information to be displayed, information indicating the subject's emotions, etc., for which the magnitude of variation fluctuation cannot be simply evaluated.

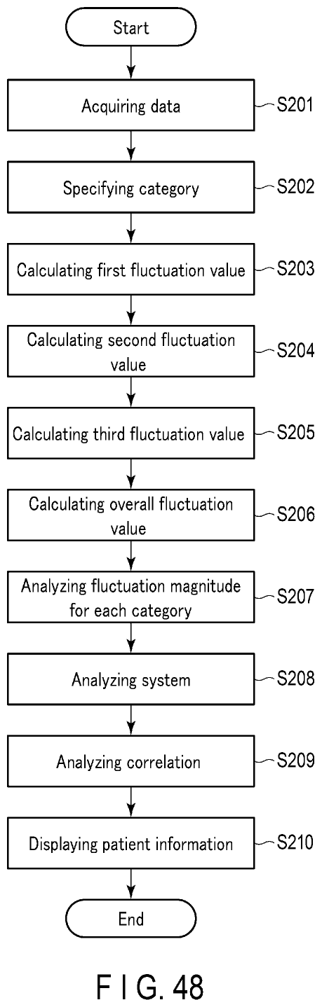

Next, the processing executed by the diagnosis support apparatus 2100 according to the present embodiment will be described. FIG. 48 is a flowchart showing an example of processing executed by the diagnosis support apparatus 2100 according to the present embodiment.

First, the acquisition function 2151 acquires medical information collected by a plurality of healthcare professionals (step S201). Specifically, the acquisition function 2151 refers to the medical database 2210 via the NW interface 2110 and acquires medical information of a target subject.

Next, the specifying function 2153 specifies a category of each item of the medical information acquired in step S201 (step S202). Specifically, the specifying function 2153 refers to the category specifying table 2121, and specifies which one of the categories of “medical indication”, “patient's view and preference”, “QOL”, and “contextual features” each item of the medical information belongs to.

Next, the calculation function 2152 calculates the first fluctuation value for each item of the medical information acquired in step S201 (step S203). For example, the calculation function 2152 quantifies each item of the medical information for each information collector and calculates a standard deviation in numerical values of the pieces of the medical information corresponding to each corresponding information collector. The calculation function 2152 then sets the calculated standard deviation to the first fluctuation value.

Next, the calculation function 2152 calculates the second fluctuation value for each item of the medical information acquired in step S201 (step S204). For example, the calculation function 2152 quantifies each item of the medical information for each date and time when the information was collected, and calculates a difference between the minimum value and the maximum value of values corresponding to the date and time when each item of the medical information was collected. The calculation function 2152 then determines, as the second fluctuation value, the calculated difference between the minimum value and the maximum value.

The calculation function 2152 then calculates the third fluctuation value for each item of the medical information acquired in step S201 (step S205). For example, the calculation function 2152 quantifies each item of the medical information for each event in which the information is collected, and calculates a standard deviation in numerical values of the pieces of the medial information corresponding to a corresponding event in which the corresponding medical information was collected. The calculation function 2152 then sets the calculated standard deviation to the third fluctuation value.

Furthermore, the calculation function 2152 sums the first fluctuation value to the third fluctuation value calculated in steps S203 to S204 and calculates the overall fluctuation value (step S206). Next, the analysis function 2154 analyzes the magnitude of a fluctuation for each category based on the overall fluctuation value calculated in step S208 (step S207).

For example, the analysis function 2154 calculates an average value of the overall fluctuation values for each of the categories of “medical indication”, “patient's view and preference”, “QOL”, and “contextual features”. The analysis function 2154 then classifies the degree of magnitude of fluctuations of “medical indication”, “patient's view and preference”, “QOL”, and “contextual features” according to the degree of the average value of the overall fluctuation values into three levels, large, medium, and small.

The analysis function 2154 then analyzes the type of fluctuation of each item of the medical information based on the first fluctuation value to the third fluctuation value calculated in steps S203 to S204 (step S208). Specifically, the analysis function 2154 determines whether or not each of the first to third fluctuation values of each item of the medical information exceeds a threshold value. The analysis function 2154 then specifies the type of a fluctuation of each item of the medical information based on the combination of determination results.

Next, the analysis function 2154 analyzes a correlation of the fluctuation in each item of the medical information based on the overall fluctuation value calculated in step S206 (step S209). Specifically, with respect to a plurality of pieces of medical information, the analysis function 2154 calculates a correlation coefficient of an index of each item of medical information index for each pair of pieces of the medical information. The analysis function 2154 determines, based on the calculated correlation coefficient, whether or not a correlation is observed between a pair of pieces of the medical information for each pair.

The display control function 2155 causes the display 2140 to display each piece of the medical information acquired in step S201, together with the overall fluctuation value calculated in step S206 (step S210), and terminates this processing. Specifically, the display control function 2155 controls the display 2140 to display a display screen as shown in FIG. 37 to FIG. 47.

As described above, with respect to a plurality of pieces of the medical information, the diagnosis support apparatus 2100 according to the present embodiment calculates a fluctuation value indicating the magnitude of variation among the pieces of the medical information, and displays information representing the fluctuation value together with the medical information.

This enables a user to grasp how much fluctuation is observed in one item of the medical information collected by a user himself or herself, as compared to medical information of the same type collected by another information collector, medical information collected on another date, medical information collected for another medical event, etc. Therefore, for example, in a situation in which an attending physician in charge of a patient determines a treatment plan for the patient according to the patient's view and preference, if there is a fluctuation in the patient's view and preference, the attending physician can notice the probability that it may not appropriate to determine the treatment plan based on the information collected by himself or herself.

Therefore, according to the diagnosis support apparatus 2100 according to the present embodiment, patient information collected from various viewpoints can be shared among the medical team and used for decision-making.

The embodiment described above can be appropriately modified and implemented by changing a part of the configuration or function of each apparatus. Thus, hereinafter, a modification of the embodiment described above will be described as another embodiment. In the following, the differences from the embodiment described above will be mainly described, and detailed descriptions of the points common to those already described will be omitted. Furthermore, the modification described below may be carried out individually or in combination as appropriate.

### Modification of Second Embodiment

The above embodiment described the mode in which the display control function 2155 displays the information collector display areas CI1 to CI3 in the temporal change display area TI. However, the display control function 2155 may display pieces of the medical information in the temporal change display area TI.

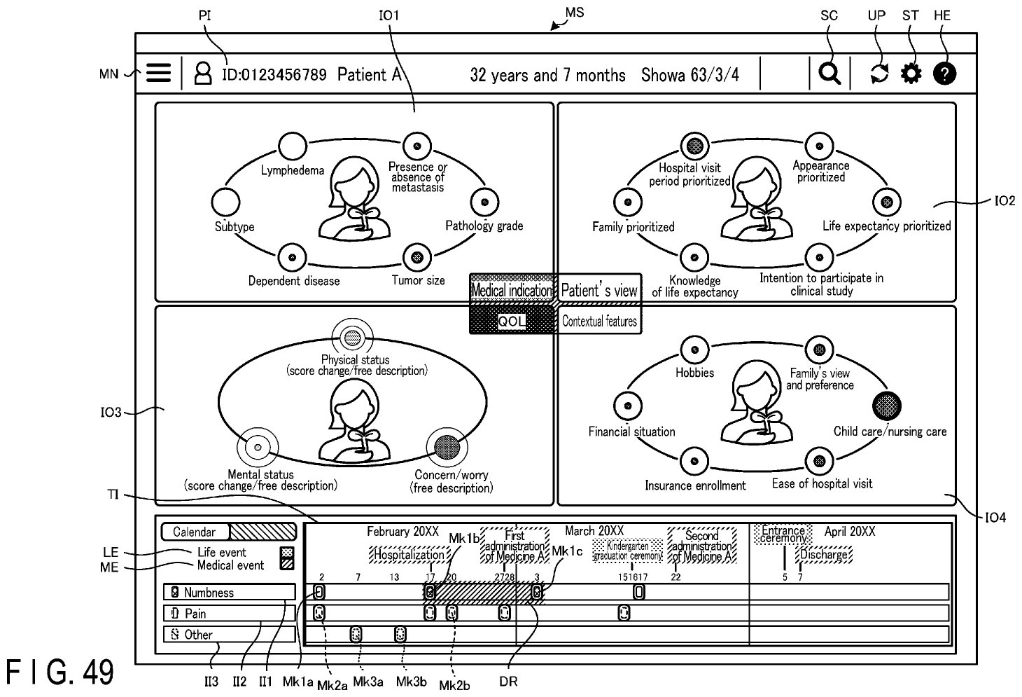

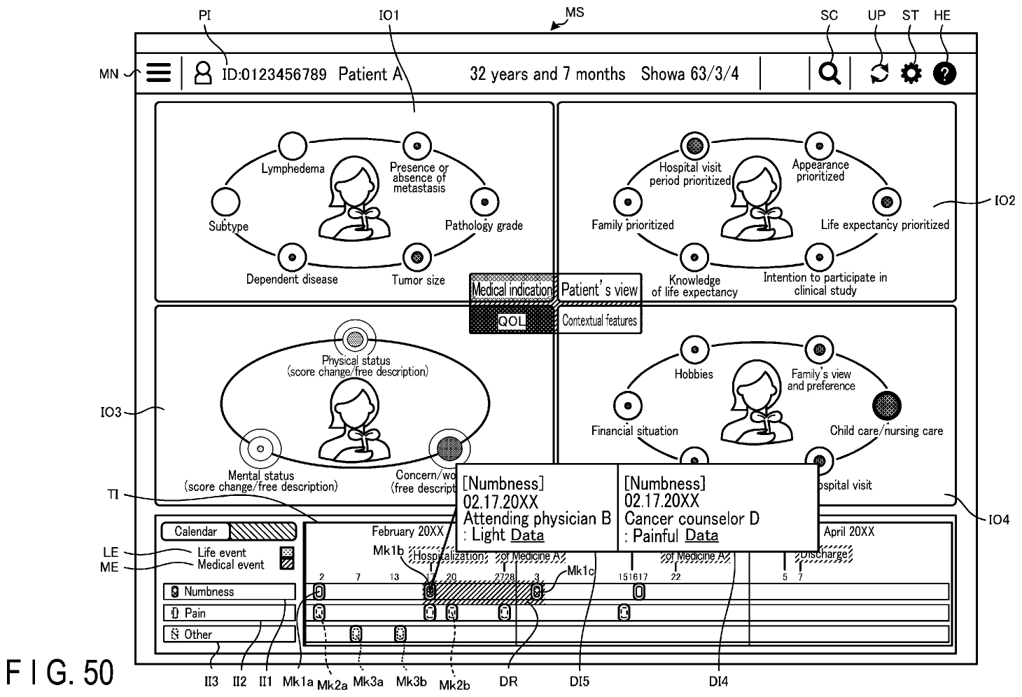

FIG. 49 and FIG. 50 each are a diagram showing an example of displaying medical information according to the modification. In FIGS. 49 and 50, medical information item display areas II1 to II3 are displayed instead of the information collector display areas CI1 to CI3. In FIGS. 49 and 50, “numbness” is displayed in the medical information item display area II1, “pain” is displayed in the medical information item display area II2, and “other” is displayed in the medical information item display area II3.

FIGS. 49 and 50 set, as the data range DR, the category of “QOL”, and the subcategories of “physical status (score change/free description)” and “numbness” acquired by each information collector on Feb. 17 and Mar. 3, 20XX. This indicates that a user is paying attention to a temporal change in “numbness” acquired by each information collector from February 17 to Mar. 3, 20XX.

FIG. 50 shows the medical information display screen MS displaying thereon a detailed display DI4 representing the evaluation made by the attending physician B on Feb. 17, 20XX, and a detailed display DI5 representing the evaluation made by the cancer counselor D on February 17.

In the detailed display DI4, “Feb. 17, 20XX” is displayed as the date and time when the medical information was collected, “attending physician B” as the information collector, and “light” as the content of the medical information. Furthermore, in the detailed display DI5, “Feb. 17, 20XX” is displayed as the date and time when the medical information was collected, “cancer counselor D” is displayed as the information collector, and “painful” is displayed as the content of the medical information.

The diagnosis support apparatus 2100 according to this modification enables a user to easily check how a fluctuation is observed in each item of the medical information.

According to at least one embodiment described above, medical-care using information on various dimensions of the patient can be supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A diagnosis support system comprising a processing circuit configured to:
acquire medical data;
calculate, based on the medical data, a feature amount in the medical data for a predetermined period; and
cause a displayer to display the medical data in a mode according to the feature amount.

2. The diagnosis support system according to claim 1, wherein the medical data includes a plurality of types of medical records acquired at a plurality of points in time; and
the processing circuit is configured to perform analysis processing on the medical data, extract, based on a result of the analysis processing, one or more descriptions related to a psychosocial item from the medical data, calculate an information amount of the psychosocial item as the feature amount using a result of extraction, and cause the displayer to display the psychosocial item in a mode according to the information amount.

3. The diagnosis support system according to claim 2, wherein the processing circuit is configured to cause the extracted descriptions to be displayed along with time-series information.

4. The diagnosis support system according to claim 2, wherein the processing circuit is configured to calculate the information amount based on a number of the extracted descriptions.

5. The diagnosis support system according to claim 2, wherein the processing circuit is configured to calculate the information amount based on a similarity between the extracted descriptions.

6. The diagnosis support system according to claim 2, wherein the processing circuit is configured to further calculate a width of a fluctuation in the psychosocial item using the result of extraction, and cause the psychosocial item to be displayed in a mode according to the width of the fluctuation.

7. The diagnosis support system according to claim 2, wherein the processing circuit is configured to further cause a temporal change in a parameter related to the psychosocial item to be displayed.

8. The diagnosis support system according to claim 2, wherein the processing circuit is mounted on a cloud server.

9. The diagnosis support system according to claim 1, wherein the processing circuit is configured to:
acquire, as the medical data, one or more pieces of medical information on a patient collected by a plurality of healthcare professionals;
calculate, for the pieces of the medical information acquired by an acquirer, a fluctuation value indicative of a magnitude of a variation between the pieces of the medical information of a same type; and
cause the displayer to display the pieces of the medical information acquired by the acquirer, for each type of the pieces of the medical information, along with information indicative of the fluctuation value calculated by the calculator.

10. The diagnosis support system according to claim 9, wherein the pieces of the medical information include subjective information of the patient input by the plurality of healthcare professionals.

11. The diagnosis support system according to claim 10, wherein the processing circuit is configured to calculate the fluctuation value based on an evaluation index for evaluating the subjective information, defined for each piece of the subjective information.

12. The diagnosis support system according to claim 9, wherein the processing circuit is configured to:
calculate a first fluctuation value indicative of a magnitude of a variation in the pieces of the medical information between the plurality of healthcare professionals who collected the pieces of the medical information; and
cause the medical information to be displayed along with information indicative of the first fluctuation value.

13. The diagnosis support system according to claim 12, wherein the processing circuit is configured to:
calculate, for the pieces of the medical information, a second fluctuation value indicative of a magnitude of a temporal variation in the pieces of the medical information; and
cause the medical information to be displayed along with information indicative of the second fluctuation value.

14. The diagnosis support system according to claim 13, wherein the processing circuit is configured to:
calculate a third fluctuation value indicative of a magnitude of a variation in the pieces of the medical information between medical events which are related to a medical treatment of the patient and from which the pieces of the medical information were collected; and
cause the pieces of the medical information to be displayed along with information indicative of the third fluctuation value.

15. The diagnosis support system according to claim 14, wherein the processing circuit is configured to specify a type of a fluctuation indicative of a variation between the pieces of the medical information from the first fluctuation value, the second fluctuation value, and the third fluctuation value each calculated by the calculator.

16. The diagnosis support system according to claim 9, wherein the processing circuit is configured to specify, from categories, a category to which a piece of the medical information belongs, based on correspondence information associating the piece of the medical information with categories of the pieces of the medial information, and cause the piece of the medical information to be displayed along with information indicative of the category to which the piece of the medical information belongs.

17. The diagnosis support system according to claim 16, wherein the categories include a medical indication, a patient's intention, a QOL, and a contextual feature; and
the processing circuit is configured to divide a display area for the medical information into four areas, an area for displaying the medical indication, an area for displaying the patient's intention, an area for displaying the QOL, and an area for displaying the contextual feature, and cause the pieces of the medical information to be displayed in the areas corresponding to the categories of the pieces of the medical information, respectively.

18. The diagnosis support system according to claim 9, wherein the processing circuit is configured to analyze a correlation between the pieces of the medical information by calculating a correlation coefficient of indexes related to the pieces of the medical information for each pair of the pieces of the medical information, and cause information indicative of the correlation between the pieces of the medical information to be displayed.

19. The diagnosis support system according to claim 2, wherein the description is a medical record, a sentence included in the medical record, or a paragraph composed of a plurality of sentences including the sentence.

20. A diagnosis support apparatus comprising:
an acquirer configured to acquire medical data;
a calculator configured to calculate, based on the medical data, a feature amount in the medical data for a predetermined period; and
a display controller configured to cause a displayer to display the medical data in a mode according to the feature amount.
